(19) United States
(12) Patent Application Publication
(10) Pub. No.: US 2026/0217653 A1
(43) Pub. Date: Jul. 30, 2026

(54) CRYSTALLINE COMPOUND OF AN LPAR1 ANTAGONIST

(71) Applicant: CONTINEUM THERAPEUTICS, INC., San Diego, CA (US)

(72) Inventors: Jeffrey Roger ROPPE, Temecula, CA (US); Austin Chih-Yu CHEN, San Diego, CA (US); Yifeng XIONG, San Diego, CA (US); Thomas SCHRADER, San Diego, CA (US); Lino VALDEZ, San Diego, CA (US)

(21) Appl. No.: 19/148,979

(22) PCT Filed: Jan. 26, 2024
(86) PCT No.: PCT/US2024/013232

§ 371 Date: Jul. 17, 2025

Related U.S. Application Data
(60) Provisional application No. 63/481,956, filed on Jan. 27, 2023.

(51) Int. Cl. C07D 213/75; A61K 31/44

(52) U.S. Cl. CPC C07D 213/75; A61K 31/44

(57) ABSTRACT

Described herein, inter alia, a crystalline compound of an LPAR1 antagonist, pharmaceutical compositions including the crystalline compound, and methods of treatment using the same.

### CROSS REFERENCE TO RELATED APPLICATIONS

This application claims priority to U.S. Provisional Application No. 63/481,956, filed Jan. 27, 2023, which is hereby incorporated by reference in its entirety and for all purposes.

### BACKGROUND

Lysophosphatidic acid, or LPA, is a family of bioactive phospholipids that are associated with multiple cellular functions. While the family members differ with regards to the length and the degree of saturation of their respective long-chain fatty acid backbone (Fujiwara et al., J. Biol. Chem., 2005, 280, 35038-35050), they are all capped by a glycerol-phosphate group through an ester linkage. LPAs are produced biologically from membrane phospholipids through a multi-step cascade mediated by enzymes that include lysophospholipase D (lysoPLD), autotaxin (ATX), phospholipase A1 (PLA1), phospholipase A2 (PLA2) and acylglycerol kinase (AGK) (Mutoh et al., British J Pharmacol., 2012, 165, 829-844). Once formed, the LPAs can regulate numerous cellular signaling pathways by binding to a class of 7-membrane domain G protein-coupled receptors (GPCRs), collectively known as LPA receptors (LPARs), of which six have been characterized: LPAR1, LPAR2, LPAR3, LPAR4, LPAR5, and LPAR6 (Choi, J. W., Annu. Rev. Pharmacol. Toxicol., 2010, 50, 157-186). The biological responses elicited by the binding of LPAs to LPARs are both wide-ranging and context-dependent (Yung et al., J. Lipid Res. 2014, 55, 1192-1214; Yung et al., Neuron 2015, 85, 669-682). These can include induction of cell proliferation, stimulation of cell migration and contraction, promotion of neurite retraction, suppression of apoptosis, initiation of chemotaxis, closure of gap junction, and others (Chun et al., Editors, Lysophospholipid Receptors: Signaling and Biochemistry, 2013, Wiley, ISBN: 978-0-470-56905-4). Furthermore, aberrant upregulation of the LPA pathway has been implicated in multiple diseases, including cancer, inflammatory diseases, infertility, neuropathic pain, psychotic and neurodegenerative disorders, atherosclerosis, as well as fibrosis of the skin, kidney, lung, and liver (Choi, J. W., Annu. Rev. Pharmacol. Toxicol., 2010, 50, 157-186: Noguchi et al., Curr. Opin Pharmacol., 2009, 9, 15-23; Yanagida et al., J Biochem., 2011, 150, 223-232). Consequently, the targeting of LPA receptors has been, and continues to be an area of intense interest for the identification of potential treatments for these disorders. Disclosed herein, inter alia, are solutions to these and other problems in the art.

### BRIEF SUMMARY

In an aspect is provided a crystalline compound having the formula:

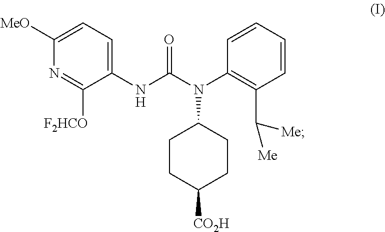 [SMILES: COc1ccc(NC(=O)N(c2ccccc2C(C)C)[C@H]2CC[C@H](C(=O)O)CC2)c(OC(F)F)n1]

wherein the crystalline compound has characteristic X-ray powder diffraction peaks at about 5.2° 2θ, about 10.4° 2θ, and about 15.6° 20.

In an aspect is provided a pharmaceutical composition including the crystalline compound described herein and a pharmaceutically acceptable excipient.

In an aspect is provided a method of treating a neurodegenerative disorder in a subject in need thereof, the method including administering to the subject in need thereof a therapeutically effective amount of the crystalline compound described herein.

In an aspect is provided a method of treating an inflammatory disease in a subject in need thereof, the method including administering to the subject in need thereof a therapeutically effective amount of the crystalline compound described herein.

In an aspect is provided a method of treating a demyelinating disease in a subject in need thereof, the method including administering to the subject in need thereof a therapeutically effective amount of the crystalline compound described herein.

In an aspect is provided a method of treating fibrotic disease in a subject in need thereof, the method including administering to the subject in need thereof a therapeutically effective amount of the crystalline compound described herein.

In an aspect is provided a method of treating cancer in a subject in need thereof, the method including administering to the subject in need thereof a therapeutically effective amount of the crystalline compound described herein.

In an aspect is provided a method of modulating LPAR1 activity in a subject, the method including administering to the subject the crystalline compound described herein.

### BRIEF DESCRIPTION OF THE DRAWINGS

FIG. 1. XRPD pattern of Pattern A crystalline polymorph.

FIG. 2. DSC thermogram of Pattern A crystalline polymorph.

FIG. 3. TGA thermogram of Pattern A crystalline polymorph.

FIG. 4. ¹H-NMR spectrum of Pattern A crystalline polymorph.

FIG. 5. PLM photograph of Pattern A crystalline polymorph.

FIG. 6. Simulated XRPD pattern of single crystal cultivated from slow evaporation.

FIG. 7. XRPD overlap of samples obtained after 1-week storage of Pattern A crystalline polymorph at 25° C./92% RH, 40° C./75% RH, and 60° C.

FIGS. 8A-8B. DVS isotherm plot and DVS isotherm analysis report of Pattern A crystalline polymorph under conditions examined.

FIG. 9. XRPD overlay of Pattern A crystalline polymorph before (bottom) and after (top) DVS test.

FIG. 10. XRPD overlay of samples obtained by compressing Pattern A crystalline polymorph at 5 MPa and 10 MPa.

FIG. 11. XRPD overlay of samples obtained by manual dry grinding of Pattern A crystalline polymorph with a mortar and pestle for 1 min, 3 min, and 5 min.

FIG. 12. XRPD overlay of samples obtained by wet grinding of Pattern A crystalline polymorph with a mortar and pestle in water and ethanol.

FIG. 13. XRPD overlay of samples obtained by equilibration at 25° C. for 2 weeks in methanol, ethanol, isopropanol, acetone, and methyl ethyl ketone.

FIG. 14. XRPD overlay of samples obtained by equilibration at 25° C. for 2 weeks in ethyl acetate, acetonitrile, IPAc, tetrahydrofuran, and 1,4-dioxane.

FIG. 15. XRPD overlay of samples obtained by equilibration at 25° C. for 2 weeks in dichloromethane, 2-MeTHF, EtOH/water, acetone/water, and MEK/water.

FIG. 16. XRPD overlay of samples obtained by equilibration at 25° C. for 2 weeks in ACN/water, 1,4-dioxane/water, THF/water, EA/heptane, and 2-MeTHF/MTBE.

FIG. 17. XRPD overlay of samples obtained by equilibration at 25° C. for 2 weeks in IPA/heptane.

FIG. 18. XRPD overlay of samples obtained by equilibration at 50° C. for 1 week in methanol, ethanol, isopropanol, acetone, and methyl ethyl ketone.

FIG. 19. XRPD overlay of samples obtained by equilibration at 50° C. for 1 week in ethyl acetate, acetonitrile, IPAc, tetrahydrofuran, and 1,4-dioxane.

FIG. 20. XRPD overlay of samples obtained by equilibration at 50° C. for 1 week in MeOH/water, 2-MeTHF, EtOH/water, acetone/water, and MEK/water.

FIG. 21. XRPD overlay of samples obtained by equilibration at 50° C. for 1 week in ACN/water, 1,4-dioxane/water, THF/water, EA/heptane, and 2-MeTHF/MTBE.

FIG. 22. XRPD overlay of samples obtained by equilibration at 50° C. for 1 week in IPA/heptane.

FIG. 23. XRPD overlay of samples obtained by equilibration at 90° C. for 5 days in 1,4-dioxane and toluene.

FIG. 24. XRPD overlay of samples obtained by equilibration under a temperature cycle in methanol, ethanol, isopropanol, acetone, and methyl ethyl ketone.

FIG. 25. XRPD overlay of samples obtained by equilibration under a temperature cycle in ethyl acetate, acetonitrile. IPAc, tetrahydrofuran, and 1,4-dioxane.

FIG. 26. XRPD overlay of samples obtained by equilibration under a temperature cycle in MeOH/water, 2-MeTHF, EtOH/water, acetone/water, and MEK/water.

FIG. 27. XRPD overlay of samples obtained by equilibration under a temperature cycle in ACN/water, 1,4-dioxane/water, THF/water, EA/heptane, and 2-MeTHF/MTBE.

FIG. 28. XRPD overlay of samples obtained by slow cooling in methanol, ethanol, isopropanol, acetone, and methyl ethyl ketone.

FIG. 29. XRPD overlay of samples obtained by slow cooling in ethyl acetate, acetonitrile, IPAc, tetrahydrofurn, and 2-MeTHF.

FIG. 30. XRPD overlay of samples obtained by fast cooling in methanol, ethanol, isopropanol, acetone, and methyl ethyl ketone.

FIG. 31. XRPD overlay of samples obtained by fast cooling in ethyl acetate, acetonitrile, IPAc, tetrahydrofurn, and 2-MeTHF.

FIG. 32. XRPD overlay of samples obtained by addition of anti-solvent in methanol/water, methyl ethyl ketone/ACN, ethyl acetate/heptane, and tetrahydrofuran/water.

FIG. 33. XRPD overlay of samples obtained by addition of anti-solvent in tetrahydrofuran/MTBE, DCM/heptane, and 2-MeTHF/heptane.

FIG. 34. XRPD overlay of samples obtained by addition of anti-solvent in ethanol/heptane, methyl ethyl ketone/ACN, and 2-MeTHF/ACN.

FIG. 35. XRPD overlay of samples obtained by reverse addition of anti-solvent in methanol/water, methyl ethyl ketone/ACN, ethyl acetate/heptane, and tetrahydrofuran/water.

FIG. 36. XRPD overlay of samples obtained by reverse addition of anti-solvent in tetrahydrofuran/MTBE, DCM/heptane, and 2-MeTHF/heptane.

FIG. 37. XRPD overlay of samples obtained by reverse addition of anti-solvent in ethanol/heptane, methyl ethyl ketone/ACN, and 2-MeTHF/ACN.

FIG. 38. XRPD overlay of samples obtained by slow evaporation in methanol, ethanol, isopropanol, acetone, and methyl ethyl ketone.

FIG. 39. XRPD overlay of samples obtained by slow evaporation in ethyl acetate, IPAc, tetrahydrofurn, 2-MeTHF, and DCM.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value. In embodiments, “about” includes the specified value (e.g., ±0.10, ±0.15, ±0.20, or ±0.25).

The term “polymorph” is used in accordance with its ordinary meaning and refers to a crystalline form of a compound.

As used herein, the term “crystalline” or “crystalline state” or “crystalline form” means having a physical state that is a regular three-dimensional array of atoms, ions, molecules, or molecular assemblies. Crystalline states have lattice arrays of building blocks that are arranged according to well-defined symmetries into unit cells that are repeated in three dimensions. In contrast, the term “amorphous” or “amorphous state” or “amorphous form” refers to a non-crystalline solid state. The physical state of a compound may be determined by techniques such as X-ray powder diffraction, polarized light microscopy and/or differential scanning calorimetry.

A compound, salt form, crystal polymorph, therapeutic agent, or other composition described herein may be referred to as being characterized by graphical data “substantially as depicted in” a figure. Such data may include, but is not limited to, X-ray powder diffraction spectra, NMR spectra, differential scanning calorimetry curves, and thermogravimetric analysis curves, among others. As is known in the art, such graphical data may provide additional technical information to further define the compound, salt form, crystal polymorph, therapeutic agent, or other composition. As is understood by one skilled in the art, such graphical representations of data may be subject to small variation, e.g., in peak relative intensities and peak positions due to factors such as variations in instrument response and variations in sample concentration and purity.

Where substituent groups are specified by their conventional chemical formulae, written from left to right, they equally encompass the chemically identical substituents that would result from writing the structure from right to left, e.g., —CH₂O— is equivalent to —OCH₂—.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold. 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “lysophosphatidic acid receptor 1 antagonist” or “LPAR1 antagonist” refers to any exogenously administered compound or agent that is capable of partially or completely inhibiting, or reversing, the effect of an agonist (e.g., lysophosphatidic acid) on the LPAR1 receptor. The term is inclusive of compounds or agents characterized or described as antagonists, partial antagonists, and negative allosteric modulators.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward lysophosphatidic acid receptor 1 (LPAR1) would preferentially inhibit LPAR1 over other lysophosphatidic acid receptors). In embodiments, an “lysophosphatidic acid receptor 1 selective compound” or “LPAR1-selective compound” refers to a compound (e.g., compound described herein) having selectivity towards lysophosphatidic acid receptor 1 (LPAR1). In embodiments, the compound (e.g., compound described herein) is about 5-fold, 10-fold, 20-fold, 30-fold, 40-fold, 50-fold, or about 100-fold more selective for lysophosphatidic acid receptor 1 (LPAR1) over one or more of LPAR2. LPAR3, LPAR4, LPAR5, or LPAR6. In embodiments, the compound (e.g., compound described herein) is at least 5-fold, 10-fold, 20-fold, 30-fold, 40-fold, 50-fold, or at least 100-fold more selective for lysophosphatidic acid receptor 1 (LPAR1) over one or more of LPAR2, LPAR3, LPAR4, LPAR5, or LPAR6.

“Patient” or “patient in need thereof” or “subject” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient: slowing in the rate of degeneration or decline; making the final point of degeneration less debilitating; improving a patient's physical or mental well-being. The treatment or amelioration of symptoms can be based on objective or subjective parameters: including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount.” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is an inflammatory disease. In embodiments, the disease is post-hemorrhagic encephalitis. In embodiments, the disease is a demyelinating disease. In embodiments, the disease is multiple sclerosis. In embodiments, the disease is a fibrotic disease. In embodiments, the disease is pulmonary fibrosis. In embodiments, the disease is idiopathic pulmonary fibrosis. In embodiments, the disease is a cancer. In embodiments, the disease is glioblastoma.

As used herein, the term “neurodegenerative disease” or “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE). Canavan disease, Cockayne syndrome, Corticobasal degeneration, Creutzfeldt-Jakob disease, frontotemporal dementia, Gerstmann-Straussler-Scheinker syndrome, Huntington's disease. HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia. Spinocerebellar ataxia (multiple types with varying characteristics), Spinal muscular atrophy, Steele-Richardson-Olszewski disease, or Tabes dorsalis.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, scleroderma, and atopic dermatitis.

As used herein, the term “demyelinating disease” refers to any disease or condition characterized by damage to the protective covering (e.g., myelin sheath) that surrounds nerve fibers (e.g., in the brain, optic nerves, or spinal cord). In embodiments, the demyelinating disease is a demyelinating disease of the central nervous system. In embodiments, the demyelinating disease is multiple sclerosis. In embodiments, the demyelinating disease is a demyelinating disease of the peripheral nervous system.

As used herein, the terms “fibrotic disease” and “fibrosis” refer to any disease or condition characterized by the formation of excess fibrous connective tissue. The formation of excess fibrous connective tissue may be in response to a reparative or reactive process. Fibrotic diseases include but are not limited to pulmonary fibrosis (e.g., idiopathic pulmonary fibrosis (IPF)), liver fibrosis (e.g., nonalcoholic steatohepatitis (NASH)), myelofibrosis, skin fibrosis (e.g., scleroderma), ocular fibrosis, mediastinal fibrosis, cardiac fibrosis, kidney fibrosis, stromal fibrosis, epidural fibrosis, epithelial fibrosis, or idiopathic fibrosis.

As used herein, the term “cardiovascular disorder” or “cardiovascular disease” is used in accordance with its plain ordinary meaning. In embodiments, cardiovascular diseases that may be treated with a compound, pharmaceutical composition, or method described herein include, but are not limited to, stroke, heart failure, hypertension, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma. Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor. e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “LPAR1-associated disease” refers to any disease or condition caused by aberrant activity or signaling of LPAR1. In embodiments, the LPAR1-associated disease is a neurodegenerative disease. In embodiments, the LPAR1-associated disease is an inflammatory disease. In embodiments, the LPAR1-associated disease is post-hemorrhagic encephalitis. In embodiments, the LPAR1-associated disease is a demyelinating disease. In embodiments, the LPAR1-associated disease is multiple sclerosis. In embodiments, the LPAR1-associated disease is a fibrotic disease. In embodiments, the LPAR1-associated disease is pulmonary fibrosis. In embodiments, the LPAR1-associated disease is idiopathic pulmonary fibrosis. In embodiments, the LPAR1-associated disease is a cancer. In embodiments, the LPAR1-associated disease is glioblastoma.

The term “drug” is used in accordance with its common meaning and refers to a substance which has a physiological effect (e.g, beneficial effect, is useful for treating a subject) when introduced into or to a subject (e.g., in or on the body of a subject or patient). A drug moiety is a radical of a drug.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., multiple sclerosis, fibrotic disease, encephalitis, or cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., multiple sclerosis, fibrotic disease, encephalitis, or cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “lysophosphatidic acid receptor” or “LPAR” refers to one or more of the family of G protein-coupled receptors for lysophosphatidic acid (LPA). In embodiments, LPAR includes LPAR1, LPAR2, LPAR3, LPAR4, LPAR5, and LPAR6.

The term “lysophosphatidic acid receptor 1” or “LPAR1” refers to a G protein-coupled receptor (including homologs, isoforms, and functional fragments thereof) that binds the lipid signaling molecule lysophosphatidic acid (LPA). The term includes any recombinant or naturally-occurring form of LPAR1 variants thereof that maintain LPAR1 activity (e.g., within at least 30%, 40%, 50%, 60%, 70%, 80%, 90%, 95%, or 100% activity compared to wildtype LPAR1). In embodiments, the LPAR1 protein encoded by the LPAR1 gene has the amino acid sequence set forth in or corresponding to Entrez 1902. UniProt Q92633, RefSeq (protein) NP 001392.2 or RefSeq (protein) NP_476500.1. In embodiments, the LPAR1 gene has the nucleic acid sequence set forth in RefSeq (mRNA) NM_001401.3 or RefSeq (mRNA) NM_057159.2. In embodiments, the amino acid sequence or nucleic acid sequence is the sequence known at the time of filing of the present application.

### II. Compounds

In an aspect is provided a crystalline compound of trans-4-(3-(2-(difluoromethoxy)-6-methoxypyridin-3-yl)-1-(2-isopropylphenyl)ureido)cyclohexane-1-carboxylic acid having the structure:

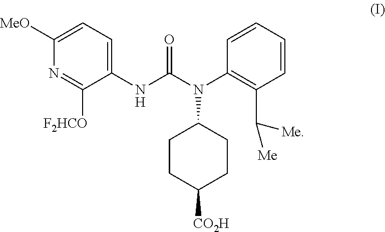 [SMILES: COc1ccc(NC(=O)N(c2ccccc2C(C)C)[C@H]2CC[C@H](C(=O)O)CC2)c(OC(F)F)n1]

In an aspect is provided a crystalline compound of having the formula:

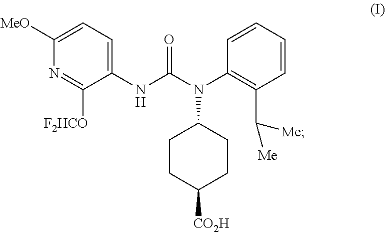 [SMILES: COc1ccc(NC(=O)N(c2ccccc2C(C)C)[C@H]2CC[C@H](C(=O)O)CC2)c(OC(F)F)n1]

wherein the crystalline compound has characteristic X-ray powder diffraction peaks at about 5.2° 2θ, about 10.4° 2θ, and about 15.6° 2θ.

In embodiments, the crystalline compound has further characteristic X-ray powder diffraction peaks at about 11.3° 2θ, about 12.6° 2θ, about 18.4° 2θ, about 20.9° 2θ, about 22.4° 2θ, about 26.1° 2θ, about 27.4° 2θ, and about 27.6° 2θ.

In embodiments, the crystalline compound has further characteristic X-ray powder diffraction peaks at about 6.7° 2θ, about 7.3° 2θ, about 8.6° 2θ, about 9.4° 2θ, about 11.3° 2θ, about 12.0° 2θ, about 12.6° 2θ, about 13.6° 2θ, about 14.9° 2θ, about 16.8° 2θ, about 17.4° 2θ, about 18.4° 2θ, about 18.8° 2θ, about 19.5° 2θ, about 20.1° 2θ, about 20.9° 2θ, about 21.8° 2θ, about 22.4° 2θ, about 22.7° 2θ, about 23.1° 2θ, about 24.1° 2θ, about 24.4° 2θ, about 24.9° 2θ, about 25.4° 2θ, about 26.1° 2θ, about 27.4° 2θ, about 27.6° 2θ, about 28.0° 2θ, about 28.4° 2θ, about 28.7° 2θ, about 29.2° 2θ, about 29.5° 2θ, about 30.0° 2θ, about 30.2° 2θ, about 31.3° 2θ, about 31.8° 2θ, about 32.0° 2θ, about 32.9° 2θ, about 33.9° 2θ, about 34.5° 2θ, about 36.3° 2θ, about 36.9° 2θ, about 37.5° 2θ, about 38.0° 2θ, and about 39.6° 2θ.

The peaks of X-ray powder diffraction pattern of the crystalline compound are provided in Table 1.

TABLE 1
XRPD peak list of the crystalline compound

| Angle | d Value | Net | Gross | Relative |
|---|---|---|---|---|
| [°2θ] | [Å] | Intensity | Intensity | Intensity [%] |
| 5.225 | 16.89908 | 20238 | 20782 | 32.7 |
| 6.652 | 13.27691 | 90.8 | 539 | 0.1 |
| 7.331 | 12.04892 | 132 | 499 | 0.2 |
| 8.633 | 10.23470 | 51.7 | 329 | 0.1 |
| 9.391 | 9.41021 | 156 | 430 | 0.3 |
| 10.404 | 8.49570 | 61945 | 62281 | 100.0 |
| 11.297 | 7.82629 | 1584 | 1940 | 2.6 |
| 11.988 | 7.37656 | 164 | 508 | 0.3 |
| 12.614 | 7.01169 | 1631 | 1944 | 2.6 |
| 13.590 | 6.51040 | 60.1 | 296 | 0.1 |
| 14.587 | 5.95791 | 89.2 | 318 | 0.1 |
| 15.618 | 5.66927 | 10808 | 11052 | 17.4 |
| 16.807 | 5.27078 | 223 | 446 | 0.4 |
| 17.420 | 5.08661 | 164 | 370 | 0.3 |
| 18.407 | 4.81622 | 883 | 1099 | 1.4 |
| 18.780 | 4.72143 | 133 | 349 | 0.2 |
| 19.512 | 4.54580 | 66.2 | 272 | 0.1 |
| 20.090 | 4.41633 | 293 | 504 | 0.5 |
| 20.868 | 4.25332 | 4545 | 4783 | 7.3 |
| 21.825 | 4.06907 | 191 | 422 | 0.3 |
| 22.430 | 3.96055 | 1046 | 1266 | 1.7 |
| 22.693 | 3.91530 | 491 | 705 | 0.8 |
| 23.136 | 3.84134 | 40.6 | 238 | 0.1 |
| 24.075 | 3.69354 | 92.5 | 275 | 0.1 |
| 24.352 | 3.65224 | 80.1 | 265 | 0.1 |
| 24.918 | 3.57048 | 269 | 472 | 0.4 |
| 25.387 | 3.50562 | 437 | 659 | 0.7 |
| 26.104 | 3.41087 | 2302 | 2542 | 3.7 |
| 27.413 | 3.25092 | 814 | 1047 | 1.3 |
| 27.566 | 3.23326 | 795 | 1024 | 1.3 |
| 28.008 | 3.18324 | 112 | 327 | 0.2 |
| 28.396 | 3.14054 | 44.3 | 253 | 0.1 |
| 28.700 | 3.10800 | 85.2 | 283 | 0.1 |
| 29.228 | 3.05308 | 163 | 351 | 0.3 |
| 29.457 | 3.02987 | 139 | 324 | 0.2 |
| 29.952 | 2.98082 | 73.5 | 253 | 0.1 |
| 30.201 | 2.95687 | 226 | 401 | 0.4 |
| 31.296 | 2.85590 | 294 | 471 | 0.5 |
| 31.766 | 2.81469 | 179 | 368 | 0.3 |
| 31.992 | 2.79528 | 337 | 528 | 0.5 |
| 32.929 | 2.71783 | 424 | 610 | 0.7 |
| 33.896 | 2.64247 | 316 | 496 | 0.5 |
| 34.514 | 2.59658 | 276 | 452 | 0.4 |
| 36.261 | 2.47537 | 96.7 | 260 | 0.2 |
| 36.897 | 2.43420 | 233 | 412 | 0.4 |
| 37.511 | 2.39574 | 161 | 337 | 0.3 |
| 38.217 | 2.35307 | 119 | 279 | 0.2 |
| 39.628 | 2.27247 | 38.9 | 184 | 0.1 |

In embodiments, the X-ray powder diffraction pattern of the crystalline compound has one or more of the peaks set forth in Table 1.

In embodiments, the crystalline compound is further characterized as having a differential scanning calorimetry endotherm onset at about 188.6° C.

In an aspect is provided a compound having the formula:

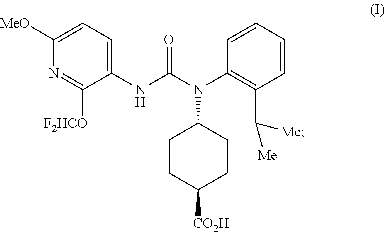 [SMILES: COc1ccc(NC(=O)N(c2ccccc2C(C)C)[C@H]2CC[C@H](C(=O)O)CC2)c(OC(F)F)n1]

wherein the compound is in a crystalline form, and the crystalline form has characteristic X-ray powder diffraction peaks at about 5.2° 2θ, about 10.4° 2θ, and about 15.6° 2θ.

In embodiments, the crystalline form has further characteristic X-ray powder diffraction peaks at about 11.3° 2θ, about 12.6° 2θ, about 18.4° 2θ, about 20.9° 2θ, about 22.4° 2θ, about 26.1° 2θ, about 27.4° 2θ, and about 27.6° 2θ.

In embodiments, the crystalline form has further characteristic X-ray powder diffraction peaks at about 6.7° 2θ, about 7.3° 2θ, about 8.6° 2θ, about 9.4° 2θ, about 11.3° 2θ, about 12.0° 2θ, about 12.6° 2θ, about 13.6° 2θ, about 14.9° 2θ, about 16.8° 2θ, about 17.4° 2θ, about 18.4° 2θ, about 18.8° 2θ, about 19.5° 2θ, about 20.1° 2θ, about 20.9° 2θ, about 21.8° 2θ, about 22.4° 2θ, about 22.7° 2θ, about 23.1° 2θ, about 24.1° 2θ, about 24.4° 2θ, about 24.9° 2θ, about 25.4° 2θ, about 26.1° 2θ, about 27.4° 2θ, about 27.6° 2θ, about 28.0° 2θ, about 28.4° 2θ, about 28.7° 2θ, about 29.2° 2θ, about 29.5° 2θ, about 30.0° 2θ, about 30.2° 2θ, about 31.3° 2θ, about 31.8° 2θ, about 32.0° 2θ, about 32.9° 2θ, about 33.9° 2θ, about 34.5° 2θ, about 36.3° 2θ, about 36.9° 2θ, about 37.5° 2θ, about 38.0° 2θ, and about 39.6° 2θ.

### III. Methods of Producing Crystalline Compound

In an aspect is provided a method of producing a crystalline compound, the method including:
(i) preparing an admixture including a solvent component and a compound having the formula:

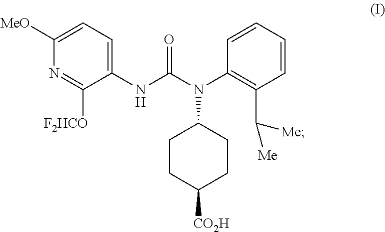 [SMILES: COc1ccc(NC(=O)N(c2ccccc2C(C)C)[C@H]2CC[C@H](C(=O)O)CC2)c(OC(F)F)n1]

and
(ii) obtaining the crystalline compound.

In embodiments, the solvent component includes one or more selected from the group consisting of methanol, ethanol, isopropanol, acetone, methyl ethyl ketone, ethyl acetate, acetonitrile, isopropyl acetate, tetrahydrofuran, 1,4-dioxane, dichloromethane, 2-methyltetrahydrofuran, water, heptane, and methyl tert-butyl ether. In embodiments, the solvent component is methanol. In embodiments, the solvent component is ethanol. In embodiments, the solvent component is isopropanol. In embodiments, the solvent component is acetone. In embodiments, the solvent component is methyl ethyl ketone. In embodiments, the solvent component is ethyl acetate. In embodiments, the solvent component is acetonitrile. In embodiments, the solvent component is isopropyl acetate. In embodiments, the solvent component is tetrahydrofuran. In embodiments, the solvent component is 1,4-dioxane. In embodiments, the solvent component is dichloromethane. In embodiments, the solvent component is 2-methyltetrahydrofuran. In embodiments, the solvent component is a mixture of ethanol and water. In embodiments, the solvent component is a mixture of methyl ethyl ketone and water. In embodiments, the solvent component is a mixture of acetonitrile and water. In embodiments, the solvent component is a mixture of 1,4-dioxane and water. In embodiments, the solvent component is a mixture of tetrahydrofuran and water. In embodiments, the solvent component is a mixture of ethyl acetate and heptane. In embodiments, the solvent component is a mixture of 2-methyltetrahydrofuran and methyl tert-butyl ether. In embodiments, the solvent component is a mixture of isopropanol and heptane.

In embodiments, the solvent component and the compound are mixed at 25° C. for two weeks. In embodiments, the solvent component and the compound are mixed at 50° C. for one week. In embodiments, the solvent component and the compound are mixed at 90° C. for five days. In embodiments, the solvent component and the compound are mixed under a temperature cycle between 5° C. and 50° C., at a cooling rate of 0.1° C. per minute, for ten cycles.

In embodiments, step (ii) includes filtering the admixture. In embodiments, step (ii) includes filtering the admixture by centrifugation. In embodiments, step (ii) includes filtering the admixture through a 0.45 μm nylon membrane filter by centrifugation. In embodiments, step (ii) includes filtering the admixture through a 0.45 μm nylon membrane filter by centrifugation at 14,000 rpm.

In embodiments, step (ii) includes slow evaporation of the solvent component.

In an aspect is provided a method of producing a crystalline compound, the method including:
(i) preparing an admixture including a solvent component and a compound having the formula:

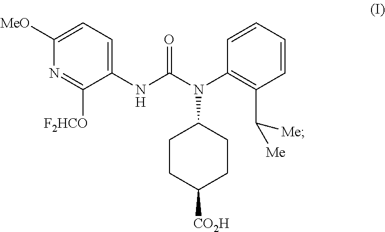 [SMILES: COc1ccc(NC(=O)N(c2ccccc2C(C)C)[C@H]2CC[C@H](C(=O)O)CC2)c(OC(F)F)n1]

(ii) heating the admixture to a first temperature;
(iii) filtering the admixture to obtain a filtrate;
(iv) cooling the filtrate to a second temperature to obtain a precipitate; and
(v) filtering the precipitate to obtain the crystalline compound.

In embodiments, the first temperature ranges from about 40° C. to about 60° C. In embodiments, the first temperature is about 40° C. In embodiments, the first temperature is about 45° C. In embodiments, the first temperature is about 50° C. In embodiments, the first temperature is about 55° C. In embodiments, the first temperature is about 60° C.

In embodiments, the second temperature ranges from about 0° C. to about 10° C. In embodiments, the second temperature is about 0° C. In embodiments, the second temperature is about 5° C. In embodiments, the first temperature is about 10° C.

In embodiments, the method further includes, after step (iv), cooling the admixture further to about −20° C.

In embodiments, the solvent component includes one or more selected from the group consisting of: methanol, ethanol, isopropanol, acetone, methyl ethyl ketone, ethyl acetate, acetonitrile, isopropyl acetate, tetrahydrofuran, and 2-methyltetrahydrofuran. In embodiments, the solvent component is methanol. In embodiments, the solvent component is ethanol. In embodiments, the solvent component is isopropanol. In embodiments, the solvent component is acetone. In embodiments, the solvent component is methyl ethyl ketone. In embodiments, the solvent component is ethyl acetate. In embodiments, the solvent component is acetonitrile. In embodiments, the solvent component is isopropyl acetate. In embodiments, the solvent component is tetrahydrofuran. In embodiments, the solvent component is 2-methyltetrahydrofuran.

In an aspect is provided a method of producing a crystalline compound, the method including:
(i) preparing an admixture including a solvent component and a compound having the formula:

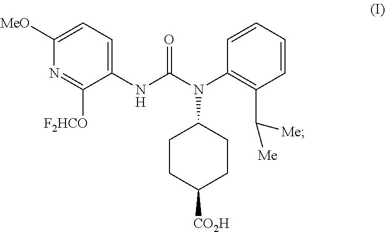 [SMILES: COc1ccc(NC(=O)N(c2ccccc2C(C)C)[C@H]2CC[C@H](C(=O)O)CC2)c(OC(F)F)n1]

(ii) filtering the admixture to obtain a filtrate;
(iii) adding an anti-solvent component to the filtrate to obtain a precipitate; and
(iv) filtering the precipitate to obtain the crystalline compound.

In embodiments, the method further includes, after step (iii), cooling the admixture to about −20° C.

In embodiments, the solvent component includes one or more selected from the group consisting of methanol, ethanol, acetone, methyl ethyl ketone, ethyl acetate, tetrahydrofuran, 1,4-dioxane, dichloromethane, and 2-methyltetrahydrofuran. In embodiments, the solvent component is methanol. In embodiments, the solvent component is ethanol. In embodiments, the solvent component is acetone. In embodiments, the solvent component is methyl ethyl ketone. In embodiments, the solvent component is ethyl acetate. In embodiments, the solvent component is tetrahydrofuran. In embodiments, the solvent component is 1,4-dioxane. In embodiments, the solvent component is dichloromethane. In embodiments, the solvent component is 2-methyltetrahydrofuran.

In embodiments, the anti-solvent component includes one or more selected from the group consisting of water, heptane, methyl tert-butyl ether, and acetonitrile. In embodiments, the anti-solvent component is water. In embodiments, the anti-solvent component is heptane. In embodiments, the anti-solvent component is methyl tert-butyl ether. In embodiments, the anti-solvent component is acetonitrile.

In an aspect is provided a method of producing a crystalline compound, the method including:
(i) preparing an admixture including a solvent component and a compound having the formula:

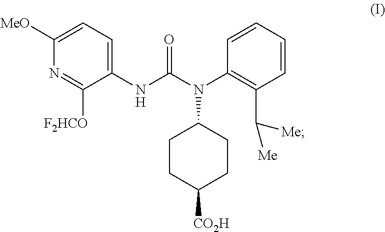 [SMILES: COc1ccc(NC(=O)N(c2ccccc2C(C)C)[C@H]2CC[C@H](C(=O)O)CC2)c(OC(F)F)n1]

(ii) filtering the admixture to obtain a filtrate;
(iii) adding the filtrate to an anti-solvent component to obtain a precipitate; and
(iv) filtering the precipitate to obtain the crystalline compound.

In embodiments, step (iii) includes adding the filtrate rapidly to the anti-solvent component.

In embodiments, the method further includes, after step (iii), cooling the admixture to about −20° C.

In embodiments, the solvent component includes one or more selected from the group consisting of methanol, ethanol, acetone, methyl ethyl ketone, ethyl acetate, tetrahydrofuran, 1,4-dioxane, dichloromethane, and 2-methyltetrahydrofuran. In embodiments, the solvent component is methanol. In embodiments, the solvent component is ethanol. In embodiments, the solvent component is acetone. In embodiments, the solvent component is methyl ethyl ketone. In embodiments, the solvent component is ethyl acetate. In embodiments, the solvent component is tetrahydrofuran. In embodiments, the solvent component is 1,4-dioxane. In embodiments, the solvent component is dichloromethane. In embodiments, the solvent component is 2-methyltetrahydrofuran.

In embodiments, the anti-solvent component includes one or more selected from the group consisting of water, heptane, methyl tert-butyl ether, and acetonitrile. In embodiments, the anti-solvent component is water. In embodiments, the anti-solvent component is heptane. In embodiments, the anti-solvent component is methyl tert-butyl ether. In embodiments, the anti-solvent component is acetonitrile.

### IV. Pharmaceutical Compositions

In an aspect is provided a pharmaceutical composition including a crystalline compound of trans-4-(3-(2-(difluoromethoxy)-6-methoxypyridin-3-yl)-1-(2-isopropylphenyl)ureido)cyclohexane-1-carboxylic acid having the structure:

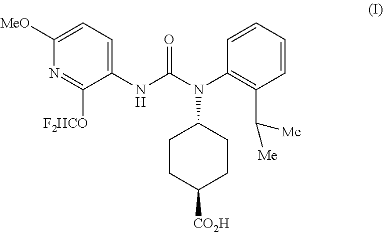 [SMILES: COc1ccc(NC(=O)N(c2ccccc2C(C)C)[C@H]2CC[C@H](C(=O)O)CC2)c(OC(F)F)n1]

and a pharmaceutically acceptable excipient.

In an aspect is provided a pharmaceutical composition including the crystalline compound described herein and a pharmaceutically acceptable excipient.

In embodiments, the pharmaceutical composition does not include the compound in a crystalline form other than the crystalline compound described herein. In embodiments, the pharmaceutical composition does not include any amorphous form of the compound.

In embodiments, the pharmaceutical composition does not include the compound in a crystalline form other than the crystalline form described herein.

In embodiments, the pharmaceutical composition includes an effective amount of the crystalline compound as described herein. In embodiments, the pharmaceutical composition includes a therapeutically effective amount of the crystalline compound as described herein.

In embodiments, the crystalline compound is further processed to provide a more uniform particle size or to control the particle size or to reduce the particle size. For example, the initial crystalline material may be subject to mechanical impact means such as crushing, grinding, milling (such as ball milling and jet milling), and the like to provide particles having the desired particle size distribution.

In embodiments, the crystalline compound described herein is substantially pure, in that it contains less than about 5%, or less than about 1%, or less than about 0.1%, of other organic small molecules, such as contaminating intermediates or by-products that are created, for example, in one or more of the steps of a synthesis method. In embodiments, the crystalline compound described herein is substantially pure, in that it contains less than about 5% of other organic small molecules, such as contaminating intermediates or by-products that are created, for example, in one or more of the steps of a synthesis method. In embodiments, the crystalline compound described herein is substantially pure, in that it contains less than about 1% of other organic small molecules, such as contaminating intermediates or by-products that are created, for example, in one or more of the steps of a synthesis method. In embodiments, the crystalline compound described herein is substantially pure, in that it contains less than about 0.1% of other organic small molecules, such as contaminating intermediates or by-products that are created, for example, in one or more of the steps of a synthesis method.

These pharmaceutical compositions include those suitable for oral, rectal, topical, buccal, parenteral (e.g., subcutaneous, intramuscular, intradermal, or intravenous), vaginal, ophthalmic, or aerosol administration.

Exemplary pharmaceutical compositions are used in the form of a pharmaceutical preparation, for example, in solid, semisolid or liquid form, which includes the crystalline compound, as an active ingredient, in a mixture with an organic or inorganic carrier or excipient suitable for external, enteral or parenteral applications. In embodiments, the crystalline compound is compounded, for example, with the usual non-toxic, pharmaceutically acceptable carriers for tablets, pellets, capsules, suppositories, solutions, emulsions, suspensions, and any other form suitable for use. The crystalline compound is included in the pharmaceutical composition in an amount sufficient to produce the desired effect upon the process or condition of the disease.

In embodiments for preparing solid compositions such as tablets, the crystalline compound is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid pre-formulation composition containing a homogeneous mixture of the crystalline compound described herein. When referring to these preformulation compositions as homogeneous, it is meant that the crystalline compound is dispersed evenly throughout the composition so that the composition is readily subdivided into equally effective unit dosage forms such as tablets, pills, and capsules.

In solid dosage forms for oral administration (e.g., capsules, tablets, pills, dragees, powders, granules and the like), the crystalline compound is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In embodiments, a tablet is made by compression or molding, optionally with one or more accessory ingredients. In embodiments, compressed tablets are prepared using binder (e.g., gelatin or hydroxypropylmethyl cellulose), lubricant, inert diluent, preservative, disintegrant (e.g., sodium starch glycolate or cross-linked sodium carboxymethyl cellulose), surface-active or dispersing agent. In embodiments, molded tablets are made by molding in a suitable machine a mixture of the crystalline compound moistened with an inert liquid diluent. In embodiments, tablets, and other solid dosage forms, such as dragees, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the crystalline compound, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol. 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In embodiments, suspensions, in addition to the crystalline compound, contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In embodiments, formulations for rectal or vaginal administration are presented as a suppository, which are prepared by mixing the crystalline compound with one or more suitable non-irritating excipients or carriers including, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of the crystalline compound include powders, sprays, ointments, pastes, creams, lotions, gels, solutions, patches and inhalants. In embodiments, the crystalline compound is mixed under sterile conditions with a pharmaceutically acceptable carrier, and with any preservatives, buffers, or propellants as required.

In embodiments, the ointments, pastes, creams and gels contain, in addition to the crystalline compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In embodiments, powders and sprays contain, in addition to the crystalline compound, excipients such as lactose, talc, silicic acid, aluminum hydroxide, calcium silicates and polyamide powder, or mixtures of these substances. In embodiments, sprays additionally contain customary propellants, such as chlorofluorohydrocarbons and volatile unsubstituted hydrocarbons, such as butane and propane.

In embodiments, the crystalline compound described herein is formulated as eye drops for ophthalmic administration.

Compositions and the crystalline compound disclosed herein alternatively are administered by aerosol. In embodiments, this is accomplished by preparing an aqueous aerosol, liposomal preparation or solid particles containing the crystalline compound. In embodiments, a non-aqueous (e.g., fluorocarbon propellant) suspension is used. In embodiments, sonic nebulizers are used. Ordinarily, an aqueous aerosol is made by formulating an aqueous solution or suspension of the crystalline compound together with conventional pharmaceutically acceptable carriers and stabilizers. The carriers and stabilizers vary with the requirements of the particular subject composition, but typically include non-ionic surfactants (e.g., Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration include the crystalline compound in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in embodiments, contain antioxidants, buffers, bacteriostats, solutes which render the formulation isotonic with the blood of the intended recipient or suspending or thickening agents.

Examples of suitable aqueous and non-aqueous carriers which are employed in the pharmaceutical compositions include water, ethanol, polyols (e.g., glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

Also contemplated are enteral pharmaceutical formulations including the crystalline compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro.

The dose of the composition including the crystalline compound described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

The crystalline compound described herein is administered to subjects or patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors, with the appropriate dosage ultimately being at the discretion of the attendant physician. For treating clinical conditions and diseases noted above, the crystalline compound disclosed herein is administered orally, subcutaneously, topically, parenterally, by inhalation spray or rectally in dosage unit formulations containing conventional non-toxic pharmaceutically acceptable carriers, adjuvants and vehicles. Parenteral administration include subcutaneous injections, intravenous or intramuscular injections or infusion techniques.

### V. Methods of Treatment

In an aspect is provided a method of treating a neurodegenerative disorder in a subject in need thereof, the method including administering to the subject in need thereof a therapeutically effective amount of a crystalline compound of trans-4-(3-(2-(difluoromethoxy)-6-methoxypyridin-3-yl)-1-(2-isopropylphenyl)ureido)cyclohexane-1-carboxylic acid having the structure:

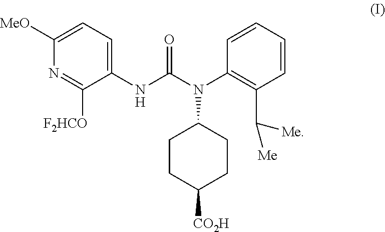 [SMILES: COc1ccc(NC(=O)N(c2ccccc2C(C)C)[C@H]2CC[C@H](C(=O)O)CC2)c(OC(F)F)n1]

In an aspect is provided a method of treating a neurodegenerative disorder in a subject in need thereof, the method including administering to the subject in need thereof a therapeutically effective amount of the crystalline compound described herein.

In an aspect is provided a method of treating an inflammatory disease in a subject in need thereof, the method including administering to the subject in need thereof a therapeutically effective amount of a crystalline compound of trans-4-(3-(2-(difluoromethoxy)-6-methoxypyridin-3-yl)-1-(2-isopropylphenyl)ureido)cyclohexane-1-carboxylic acid having the structure:

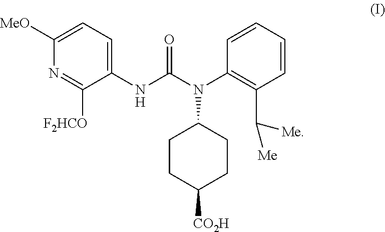 [SMILES: COc1ccc(NC(=O)N(c2ccccc2C(C)C)[C@H]2CC[C@H](C(=O)O)CC2)c(OC(F)F)n1]

In an aspect is provided a method of treating an inflammatory disease in a subject in need thereof, the method including administering to the subject in need thereof a therapeutically effective amount of the crystalline compound described herein.

In embodiments, the inflammatory disease is encephalitis. In embodiments, the inflammatory disease is post-hemorrhagic encephalitis. In embodiments, the inflammatory disease is ocular inflammation. In embodiments, the inflammatory disease is conjunctivitis. In embodiments, the inflammatory disease is allergic conjunctivitis. In embodiments, the inflammatory disease is vernal keratoconjunctivitis. In embodiments, the inflammatory disease is papillary conjunctivitis. In embodiments, the inflammatory disease is Sjogren's syndrome. In embodiments, the inflammatory disease is inflammatory disease with dry eyes.

In an aspect is provided a method of treating a demyelinating disease in a subject in need thereof, the method including administering to the subject in need thereof a therapeutically effective amount of a crystalline compound of trans-4-(3-(2-(difluoromethoxy)-6-methoxypyridin-3-yl)-1-(2-isopropylphenyl)ureido)cyclohexane-1-carboxylic acid having the structure:

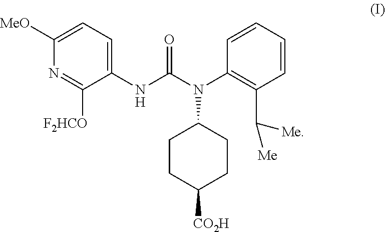 [SMILES: COc1ccc(NC(=O)N(c2ccccc2C(C)C)[C@H]2CC[C@H](C(=O)O)CC2)c(OC(F)F)n1]

In an aspect is provided a method of treating a demyelinating disease in a subject in need thereof, the method including administering to the subject in need thereof a therapeutically effective amount of the crystalline compound described herein.

In embodiments, the demyelinating disease is a demyelinating disease of the central nervous system. In embodiments, the demyelinating disease is multiple sclerosis. In embodiments, the demyelinating disease is a demyelinating disease of the peripheral nervous system.

In an aspect is provided a method of treating fibrotic disease in a subject in need thereof, the method including administering to the subject in need thereof a therapeutically effective amount of a crystalline compound of trans-4-(3-(2-(difluoromethoxy)-6-methoxypyridin-3-yl)-1-(2-isopropylphenyl)ureido)cyclohexane-1-carboxylic acid having the structure:

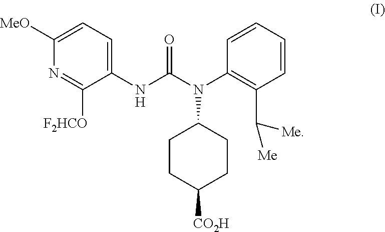 [SMILES: COc1ccc(NC(=O)N(c2ccccc2C(C)C)[C@H]2CC[C@H](C(=O)O)CC2)c(OC(F)F)n1]

In an aspect is provided a method of treating fibrotic disease in a subject in need thereof, the method including administering to the subject in need thereof a therapeutically effective amount of the crystalline compound described herein.

In embodiments, the fibrotic disease is pulmonary fibrosis. In embodiments, the fibrotic disease is skin fibrosis. In embodiments, the fibrotic disease is liver fibrosis. In embodiments, the fibrotic disease is ocular fibrosis. In embodiments, the fibrotic disease is idiopathic pulmonary fibrosis. In embodiments, the fibrotic disease is scleroderma. In embodiments, the fibrotic disease is nonalcoholic steatohepatitis. In embodiments, the fibrotic disease is ocular fibrosis. In embodiments, the fibrotic disease is hypertrophic scarring or keloids (e.g., burn induced or surgical, sarcoidosis, scleroderma, spinal cord injury/fibrosis, myelofibrosis, vascular restenosis, atherosclerosis, arteriosclerosis, Wegener's granulomatosis, mixed connective tissue disease, or Peyronie's disease). In embodiments, the fibrotic disease is iatrogenic pulmonary fibrosis. In embodiments, the fibrotic disease is radiation-induced fibrosis. In embodiments, the fibrotic disease is silicosis-induced pulmonary fibrosis. In embodiments, the fibrotic disease is asbestos-induced pulmonary fibrosis. In embodiments, the fibrotic disease is pleural fibrosis. In embodiments, the fibrotic disease is pulmonary fibrosis associated with SARS-CoV-2 infection and/or COVID-19. In embodiments, the fibrotic disease is pulmonary fibrosis secondary to systemic inflammatory disease. In embodiments, the fibrotic disease is pulmonary fibrosis secondary to sarcoidosis. In embodiments, the fibrotic disease is gut fibrosis. In embodiments, the fibrotic disease is head and neck fibrosis. In embodiments, the fibrotic disease is cirrhosis. In embodiments, the fibrotic disease is alcohol-induced liver fibrosis. In embodiments, the fibrotic disease is endometriosis. In embodiments, the fibrotic disease is spinal cord fibrosis. In embodiments, the fibrotic disease is myelofibrosis. In embodiments, the fibrotic disease is cardiac fibrosis. In embodiments, the fibrotic disease is perivascular fibrosis. In embodiments, the fibrotic disease is Peyronie's disease. In embodiments, the fibrotic disease is abdominal or bowel adhesions. In embodiments, the fibrotic disease is bladder fibrosis. In embodiments, the fibrotic disease is fibrosis of the nasal passages. In embodiments, the fibrotic disease is fibrosis mediated by fibroblasts. In embodiments, the fibrotic disease is renal fibrosis associated with chronic kidney disease (CKD).

In an aspect is provided a method of treating cancer in a subject in need thereof, the method including administering to the subject in need thereof a therapeutically effective amount of a crystalline compound of trans-4-(3-(2-(difluoromethoxy)-6-methoxypyridin-3-yl)-1-(2-isopropylphenyl)ureido)cyclohexane-1-carboxylic acid having the structure:

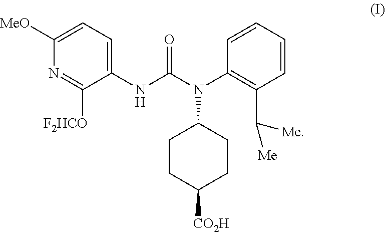 [SMILES: COc1ccc(NC(=O)N(c2ccccc2C(C)C)[C@H]2CC[C@H](C(=O)O)CC2)c(OC(F)F)n1]

In an aspect is provided a method of treating cancer in a subject in need thereof, the method including administering to the subject in need thereof a therapeutically effective amount of the crystalline compound described herein.

In embodiments, the cancer is brain cancer. In embodiments, the cancer is glioblastoma. In embodiments, the cancer is a solid tumor (e.g., of the bladder, bowel, brain, breast, endometrium, heart, kidney, lung, lymphatic tissue (e.g., lymphoma), ovary, pancreas or other endocrine organ (e.g., thyroid), prostate, skin (e.g., melanoma or basal cell cancer)) or hematological tumors (e.g., leukemia) at any stage of the disease with or without metastases. In embodiments, the cancer is acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (e.g., osteosarcoma or malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-cell lymphoma, embryonal tumors, endometrial cancer, ependymoblastoma, ependymoma, esophageal cancer, Ewing sarcoma family of tumors, eye cancer, retinoblastoma, gallbladder cancer, gastrointestinal carcinoid tumor, gastrointestinal stromal tumor (GIST), germ cell tumor, glioma, hairy cell leukemia, head and neck cancer, hepatocellular cancer, Hodgkin lymphoma, hypopharyngeal cancer, intraocular melanoma, islet cell tumors (e.g., endocrine pancreas), Kaposi's sarcoma, kidney cancer, Langerhans cell histiocytosis, laryngeal cancer, leukemia, liver cancer, lymphoma, medulloblastoma, medulloepithelioma, melanoma, mesothelioma, mouth cancer, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, stomach (e.g., gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, or Wilms' tumor.

In an aspect is provided a method of treating an LPAR1-associated disease in a subject in need thereof, the method including administering to the subject in need thereof a therapeutically effective amount of a crystalline compound of trans-4-(3-(2-(difluoromethoxy)-6-methoxypyridin-3-yl)-1-(2-isopropylphenyl)ureido)cyclohexane-1-carboxylic acid having the structure:

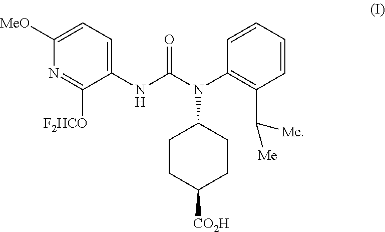 [SMILES: COc1ccc(NC(=O)N(c2ccccc2C(C)C)[C@H]2CC[C@H](C(=O)O)CC2)c(OC(F)F)n1]

In an aspect is provided a method of treating an LPAR1-associated disease in a subject in need thereof, the method including administering to the subject in need thereof a therapeutically effective amount of the crystalline compound described herein.

In embodiments, the LPAR1-associated disease is a neurodegenerative disease. In embodiments, the LPAR1-associated disease is an inflammatory disease. In embodiments, the LPAR1-associated disease is post-hemorrhagic encephalitis. In embodiments, the LPAR1-associated disease is a demyelinating disease. In embodiments, the LPAR1-associated disease is multiple sclerosis. In embodiments, the LPAR1-associated disease is a fibrotic disease. In embodiments, the LPAR1-associated disease is pulmonary fibrosis. In embodiments, the LPAR1-associated disease is idiopathic pulmonary fibrosis. In embodiments, the LPAR1-associated disease is ocular fibrosis. In embodiments, the LPAR1-associated disease is cancer (e.g., brain cancer, ovarian cancer, colon cancer, prostate cancer, breast cancer, melanoma, head and neck cancer, bowel cancer, colorectal cancer, or thyroid cancer). In embodiments, the LPAR1-associated disease is pain (e.g., neuropathic pain, acute pain, or chronic pain). In embodiments, the LPAR1-associated disease is neuropathic pain. In embodiments, the LPAR1-associated disease is acute pain. In embodiments, the LPAR1-associated disease is chronic pain.

In embodiments, the LPAR1-associated disease is a respiratory or allergic disorder. In embodiments, the respiratory or allergic disorder is asthma, peribronchiolar fibrosis, obliterative bronchiolitis, or chronic obstructive pulmonary disease (COPD). In embodiments, the COPD is chronic bronchitis or emphysema, pulmonary hypertension, interstitial lung fibrosis and/or airway inflammation, or cystic fibrosis. In embodiments, the respiratory disease is adult respiratory distress syndrome or allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, and hypoxia.

In embodiments, the LPAR1-associated disease is a nervous system disorder. In embodiments, the nervous system disorder is Alzheimer's Disease, cerebral edema, cerebral ischemia, stroke, multiple sclerosis, neuropathies, Parkinson's Disease, a nervous condition found after blunt or surgical trauma (including post-surgical cognitive dysfunction and spinal cord or brain stem injury), degenerative disk disease, or sciatica.

In embodiments, the LPAR1-associated disease is a cardiovascular disorder. In embodiments, the cardiovascular disorder is arrhythmia (e.g., atrial or ventricular); atherosclerosis and its sequelae; angina; cardiac rhythm disturbances; myocardial ischemia; myocardial infarction; cardiac or vascular aneurysm; vasculitis; stroke; peripheral obstructive arteriopathy of a limb, an organ, or a tissue; reperfusion injury following ischemia of the brain, heart or other organ or tissue; endotoxic, surgical, or traumatic shock: hypertension; valvular heart disease; heart failure; abnormal blood pressure; shock; vasoconstriction (including that associated with migraines); vascular abnormality, or a cardiovascular insufficiency limited to a single organ or tissue.

In embodiments, the LPAR1-associated disease is lung fibrosis, kidney fibrosis, liver fibrosis, scarring, asthma, rhinitis, chronic obstructive pulmonary disease (COPD), pulmonary hypertension, interstitial lung fibrosis, arthritis, allergy, psoriasis, inflammatory bowel disease, adult respiratory distress syndrome, myocardial infarction, aneurysm, stroke, cancer, pain, proliferative disorders, or inflammatory conditions.

In embodiments, the LPAR1-associated disease is a liver disease. In embodiments, the liver disease is hepatitis C, liver cancer, familial combined hyperlipidemia, non-alcoholic fatty liver disease (NAFLD), progressive familial intrahepatic cholestasis, primary biliary cirrhosis (PBC), or primary sclerosing cholangitis (PSC). In embodiments, the liver disease is primary sclerosing cholangitis (PSC). In embodiments, the liver disease includes portal hypertension. In embodiments, liver cancer includes hepatocellular carcinoma (HCC), cholangiocarcinoma, angiosarcoma, or hemangiosarcoma. In embodiments, NAFLD includes steatosis. In embodiments, NAFLD includes NASH. In embodiments, NAFLD or NASH includes liver fibrosis. In embodiments, NAFLD or NASH includes liver cirrhosis. In embodiments. NAFLD or NASH includes compensated liver cirrhosis. In embodiments, NAFLD or NASH includes decompensated liver fibrosis. In embodiments, NAFLD includes hepatocellular carcinoma (HCC). In embodiments, the liver disease is NASH.

In an aspect is provided a method of modulating LPAR1 activity in a subject, the method including administering to the subject a crystalline compound of trans-4-(3-(2-(difluoromethoxy)-6-methoxypyridin-3-yl)-1-(2-isopropylphenyl)ureido)cyclohexane-1-carboxylic acid having the structure:

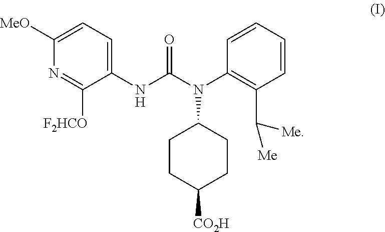 [SMILES: COc1ccc(NC(=O)N(c2ccccc2C(C)C)[C@H]2CC[C@H](C(=O)O)CC2)c(OC(F)F)n1]

In an aspect is provided a method of modulating LPAR1 activity in a subject, the method including administering to the subject the crystalline compound described herein.

### VI. Embodiments

Embodiment 1. A crystalline compound having the formula:

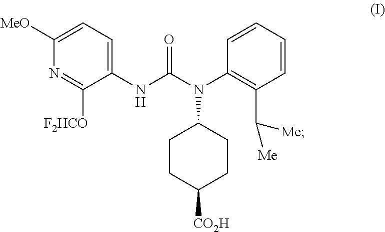 [SMILES: COc1ccc(NC(=O)N(c2ccccc2C(C)C)[C@H]2CC[C@H](C(=O)O)CC2)c(OC(F)F)n1]

wherein the crystalline compound has characteristic X-ray powder diffraction peaks at about 5.2° 2θ, about 10.4° 2θ, and about 15.6° 2θ.

Embodiment 2. The crystalline compound of embodiment 1, wherein the crystalline compound has further characteristic X-ray powder diffraction peaks at about 11.3° 2θ, about 12.6° 2θ, about 18.4° 2θ, about 20.9° 2θ, about 22.4° 2θ, about 26.1° 2θ, about 27.4° 2θ, and about 27.6° 2θ.

Embodiment 3. The crystalline compound of embodiment 1, wherein the crystalline compound has further characteristic X-ray powder diffraction peaks at about 6.7° 2θ, about 7.3° 2θ, about 8.6° 2θ, about 9.4° 2θ, about 11.3° 2θ, about 12.0° 2θ, about 12.6° 2θ, about 13.6° 2θ, about 14.9° 2θ, about 16.8° 2θ, about 17.4° 2θ, about 18.4° 2θ, about 18.8° 2θ, about 19.5° 2θ, about 20.1° 2θ, about 20.9° 2θ, about 21.8° 2θ, about 22.4° 2θ, about 22.7° 2θ, about 23.1° 2θ, about 24.1° 2θ, about 24.4° 2θ, about 24.9° 2θ, about 25.4° 2θ, about 26.1° 2θ, about 27.4° 2θ, about 27.6° 2θ, about 28.0° 2θ, about 28.4° 2θ, about 28.7° 2θ, about 29.2° 2θ, about 29.5° 2θ, about 30.0° 2θ, about 30.2° 2θ, about 31.3° 2θ, about 31.8° 2θ, about 32.0° 2θ, about 32.9° 2θ, about 33.9° 2θ, about 34.5° 2θ, about 36.3° 2θ, about 36.9° 2θ, about 37.5° 2θ, about 38.0° 2θ, and about 39.6° 2θ.

Embodiment 4. The crystalline compound of one of embodiments 1 to 3, wherein the crystalline compound is further characterized as having a differential scanning calorimetry endotherm onset at about 188.6° C.

Embodiment 5. A pharmaceutical composition comprising the crystalline compound of one of embodiments 1 to 4 and a pharmaceutically acceptable excipient.

Embodiment 6. The pharmaceutical composition of embodiment 5, wherein the pharmaceutical composition does not comprise the compound of formula I in a crystalline form other than said crystalline compound.

Embodiment 7. A method of treating a neurodegenerative disorder in a subject in need thereof, said method comprising administering to the subject in need thereof a therapeutically effective amount of the crystalline compound of one of embodiments 1 to 4.

Embodiment 8. A method of treating an inflammatory disease in a subject in need thereof, said method comprising administering to the subject in need thereof a therapeutically effective amount of the crystalline compound of one of embodiments 1 to 4.

Embodiment 9. The method of embodiment 8, wherein the inflammatory disease is encephalitis.

Embodiment 10. The method of embodiment 9, wherein the encephalitis is post-hemorrhagic encephalitis.

Embodiment 11. A method of treating a demyelinating disease in a subject in need thereof, said method comprising administering to the subject in need thereof a therapeutically effective amount of the crystalline compound of one of embodiments 1 to 4.

Embodiment 12. The method of embodiment 11, wherein the demyelinating disease is a demyelinating disease of the central nervous system.

Embodiment 13. The method of embodiment 12, wherein the demyelinating disease is multiple sclerosis.

Embodiment 14. The method of embodiment 11, wherein the demyelinating disease is a demyelinating disease of the peripheral nervous system.

Embodiment 15. A method of treating a fibrotic disease in a subject in need thereof, said method comprising administering to the subject in need thereof a therapeutically effective amount of the crystalline compound of one of embodiments 1 to 4.

Embodiment 16. The method of embodiment 15, wherein the fibrotic disease is pulmonary fibrosis, skin fibrosis, liver fibrosis, or ocular fibrosis.

Embodiment 17. The method of embodiment 15, wherein the fibrotic disease is idiopathic pulmonary fibrosis, scleroderma, nonalcoholic steatohepatitis, or ocular fibrosis.

Embodiment 18. A method of treating cancer in a subject in need thereof, said method comprising administering to the subject in need thereof a therapeutically effective amount of the crystalline compound of one of embodiments 1 to 4.

Embodiment 19. The method of embodiment 18, wherein the cancer is brain cancer.

Embodiment 20. The method of embodiment 19, wherein the cancer is glioblastoma.

Embodiment 21. A method of treating an LPAR1-associated disease in a subject in need thereof, said method comprising administering to the subject in need thereof a therapeutically effective amount of the crystalline compound of one of embodiments 1 to 4, wherein the LPAR1-associated disease is neuropathic pain.

Embodiment 22. A method of modulating LPAR1 activity in a subject, said method comprising administering to the subject the crystalline compound of one of embodiments 1 to 4.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Polymorph Screening

The polymorph screening was performed with the free acid form of trans-4-(3-(2-(difluoromethoxy)-6-methoxypyridin-3-yl)-1-(2-isopropylphenyl)ureido)cyclohexane-1-carboxylic acid. Polymorphic behaviors of the free acid form were investigated by equilibration, slow cooling, fast cooling, slow evaporation, antisolvent addition, and reverse antisolvent addition experiments.

Although about 100 polymorph screening experiments were conducted in 15 solvents and their combinations with different crystallization methods, only one crystalline polymorph, named as Pattern A, was identified.

### Characterization of Crystalline Compound

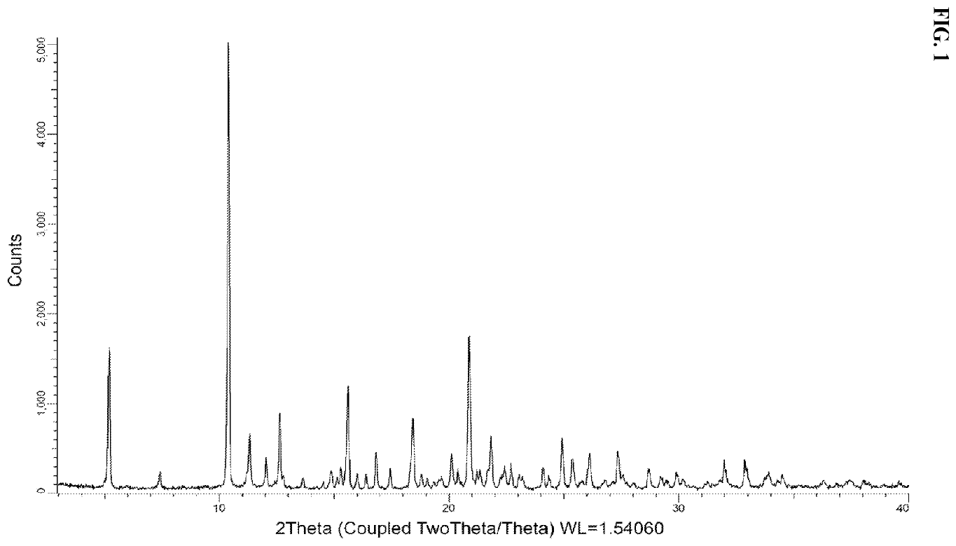

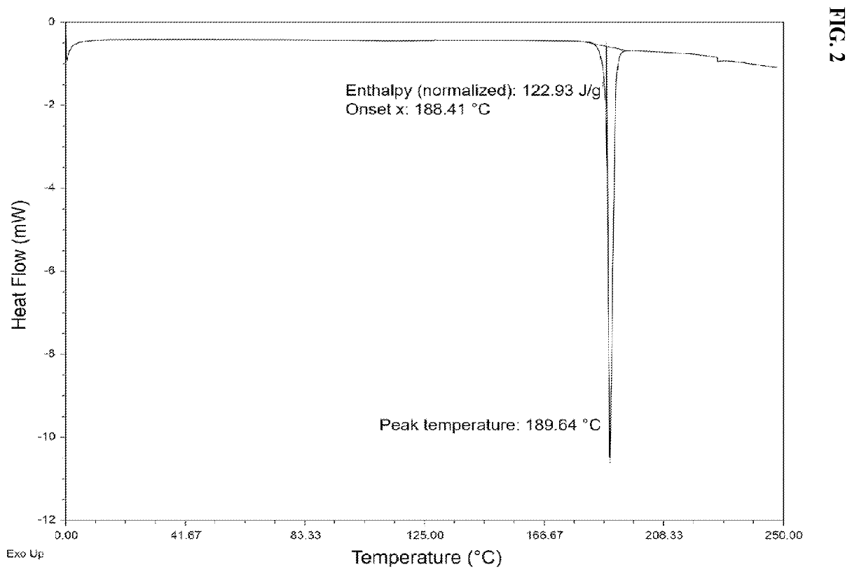

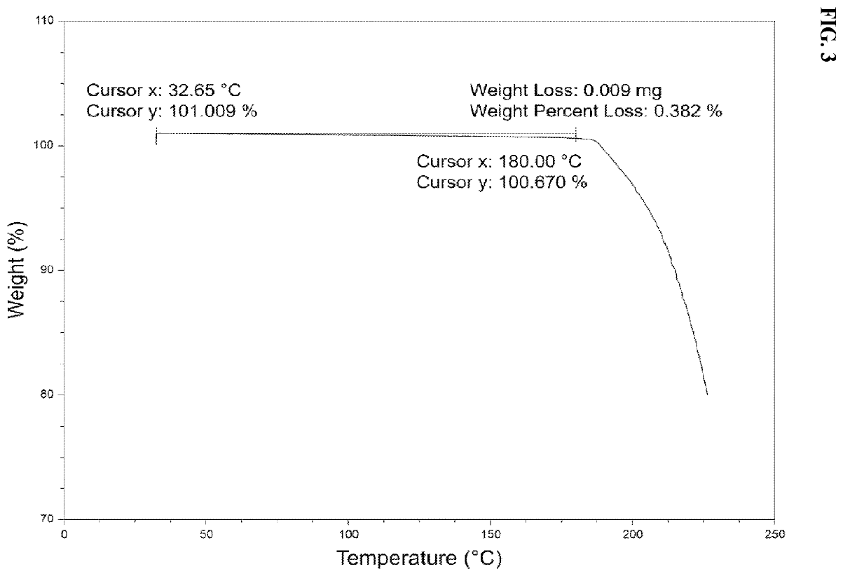

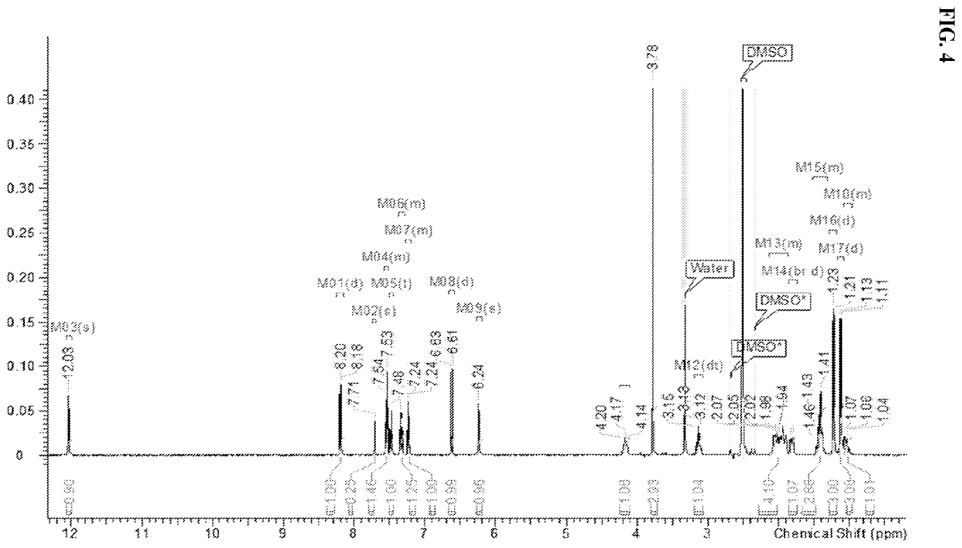

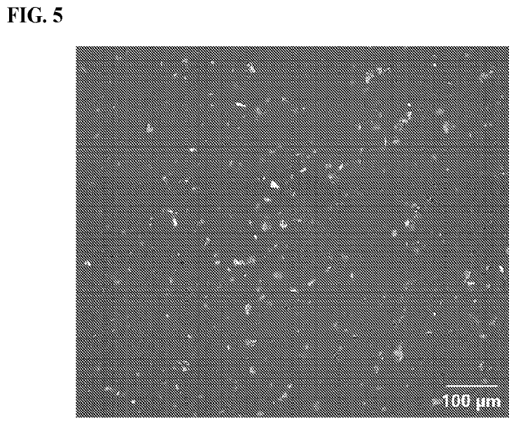

Pattern A is an anhydrate. It was obtained from most of solvent systems by equilibration, slow cooling, fast cooling, antisolvent addition, and reverse antisolvent addition experiments. Pattern A is of high crystallinity (FIG. 1). DSC shows a melting peak at Tₒₙₛₑₜ of 188.6° C. (FIG. 2). Decomposition occurs upon melting. TGA shows about 0.4% weight loss at about 180° C. (FIG. 3). ¹H-NMR shows no detectable residual solvent (FIG. 4). PLM reveals the crystal morphology to be plate-late, with particle size ranging from 1 to 20 μm (FIG. 5). The Pattern A is a stable polymorph.

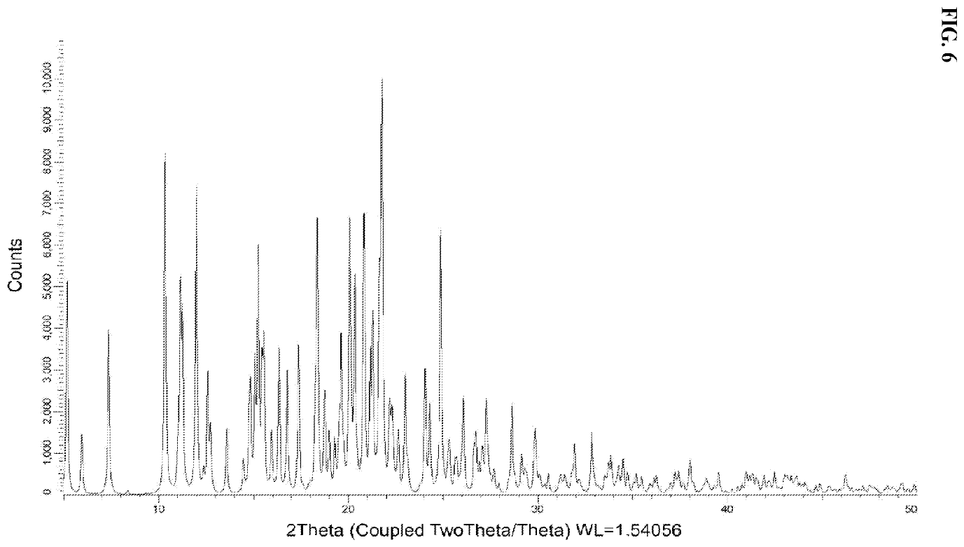

Compared with the typical XRPD pattern of Pattern A and single crystal data of Pattern A (FIG. 6), some screening samples obtained from equilibration, fast cooling, and slow evaporation contain one small extra peak at 10.0° 2θ, which could be caused by some unknown impurities.

### Bulk Stability of Pattern A

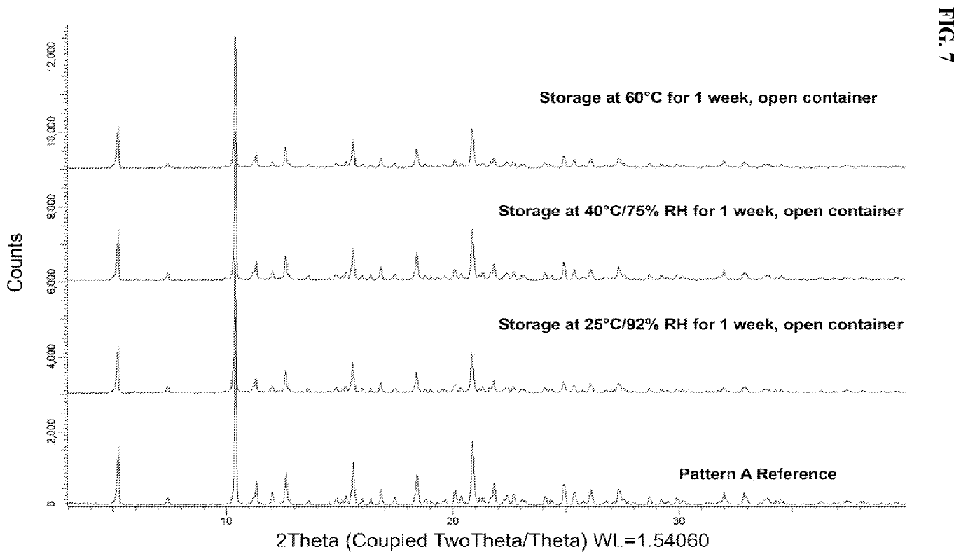

Bulk stability of Pattern A was evaluated at 25° C./92% RH in an open container, at 40° C./75% RH in an open container, and at 60° C. in a tight container over 1 week. Pattern A was found to be physically and chemically stable under these conditions over 1 week (FIG. 7).

### Hygroscopicity of Pattern A

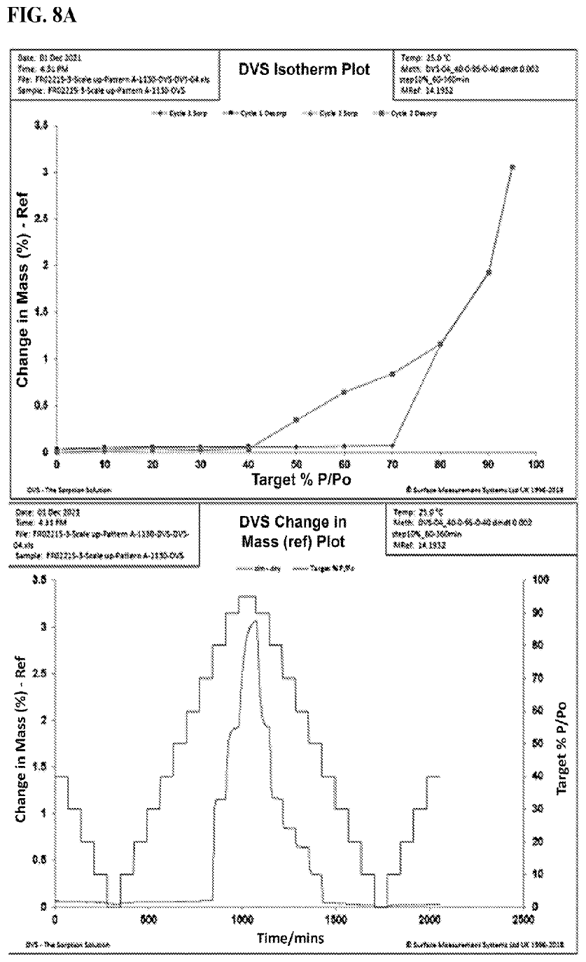

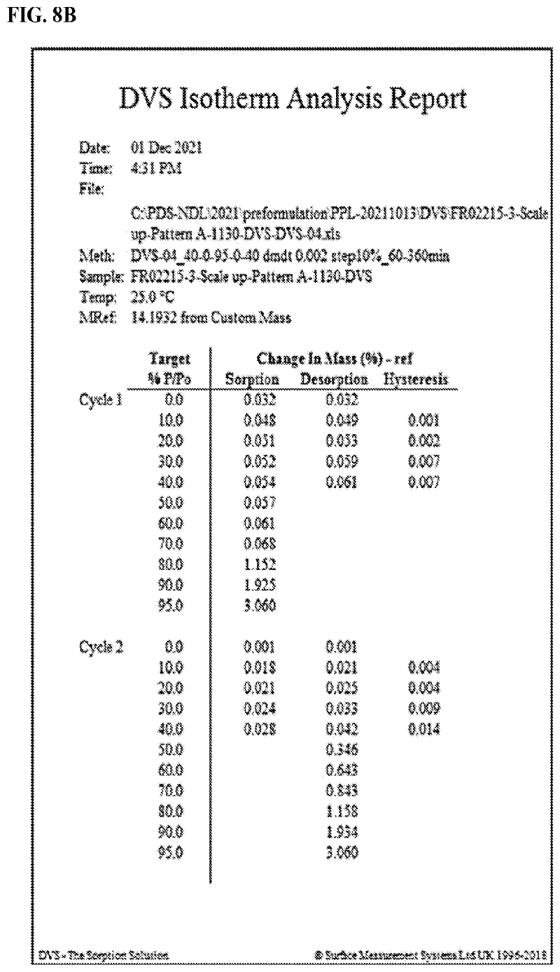

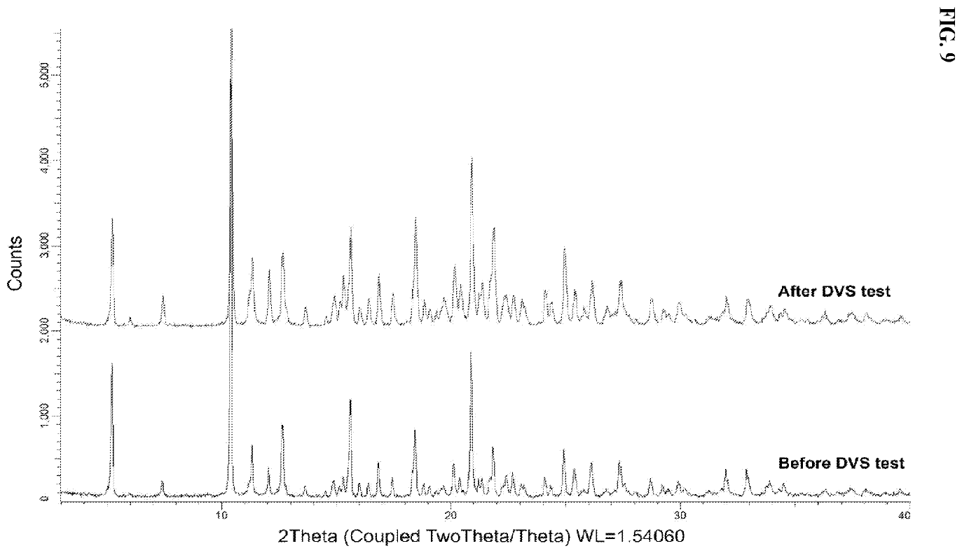

Hygroscopicity of Pattern A was evaluated by dynamic vapor sorption (DVS) test at 25° C. with a cycle of 40→0→95→0→40% RH at a rate of change in mass per time unit of 0.002% per minute. Pattern A was found to be slightly hygroscopic at 80% RH and hygroscopic at 95% RH. It absorbed about 1.2% water at 80% RH and 3.1% water at 95% RH at 25° C. (FIG. 8). After the DVS test, obtained sample was still Pattern A (FIG. 9).

### Feasibility of Formulation Process for Pattern A

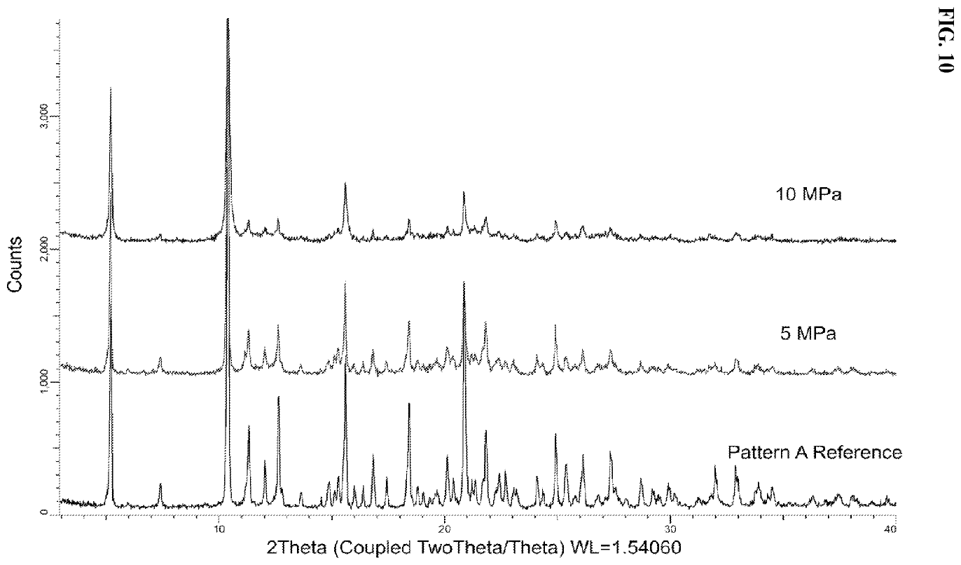

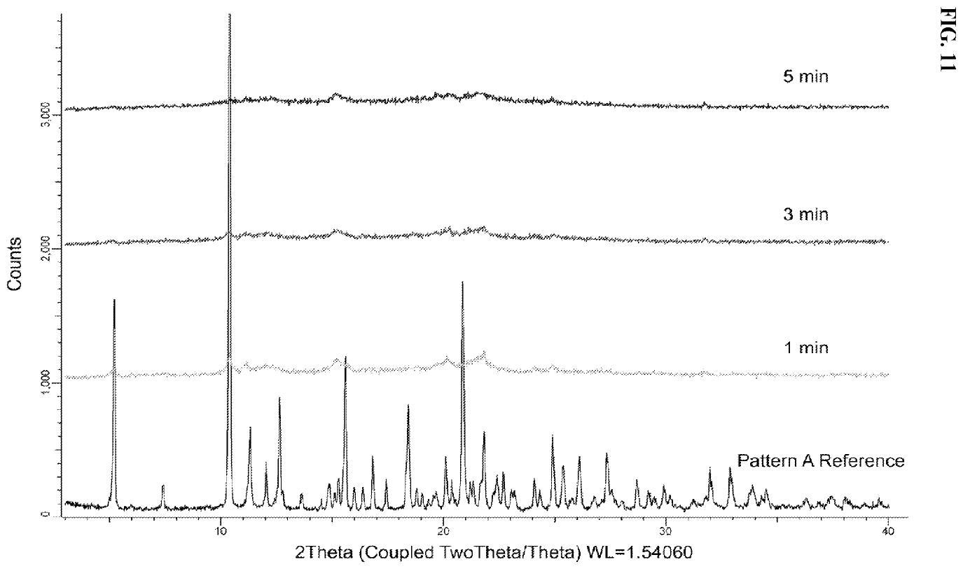

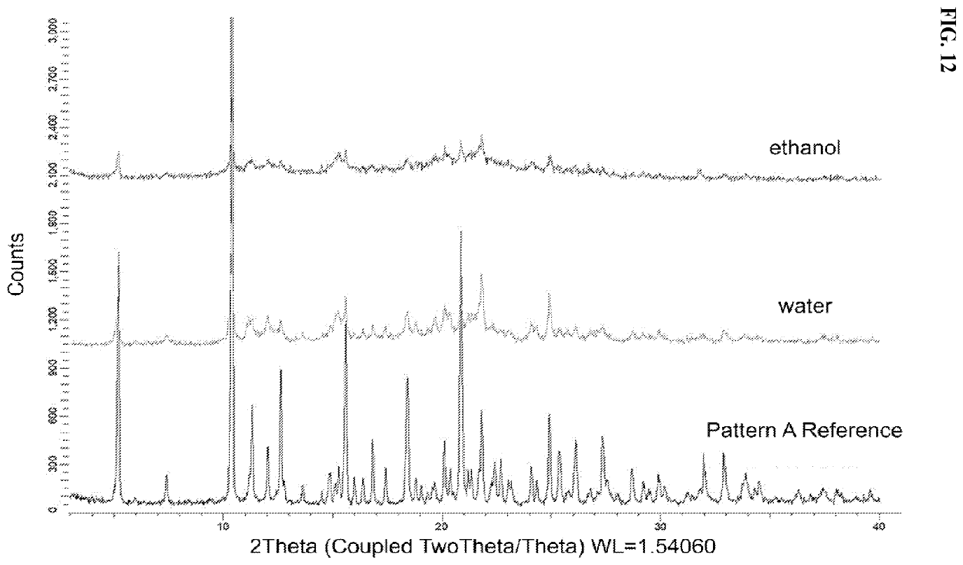

Feasibility of formulation process for Pattern A was evaluated by compression, grinding, and granulation simulation experiments. Pattern A showed good tolerance to compression with no form change and only slight crystallinity decrease (FIG. 10). Upon manual grinding, the Pattern A rapidly lost crystallinity and converted to practically amorphous form after 3 minutes of grinding with a mortar and pestle (FIG. 11). Similarly, upon wet granulation with ethanol or water, the crystallinity of Pattern A decreased significantly and partial amorphization was observed (FIG. 12).

### Polymorph Screening Experiments

### Equilibration at 25° C. For 2 Weeks

Approximately 50 mg of trans-4-(3-(2-(difluoromethoxy)-6-methoxypyridin-3-yl)-1-(2-isopropylphenyl)ureido)cyclohexane-1-carboxylic acid was equilibrated in 0.4-1.0 mL of solvents at 25° C. for 2 weeks with a stirring bar on a magnetic stirring plate at a rate of 300-400 rpm.

The obtained suspensions were then filtered through a 0.45 μm nylon membrane filter by centrifugation at 14.000 rpm. Solid parts (wet cakes) were investigated by XRPD. Results are summarized in Table 2.

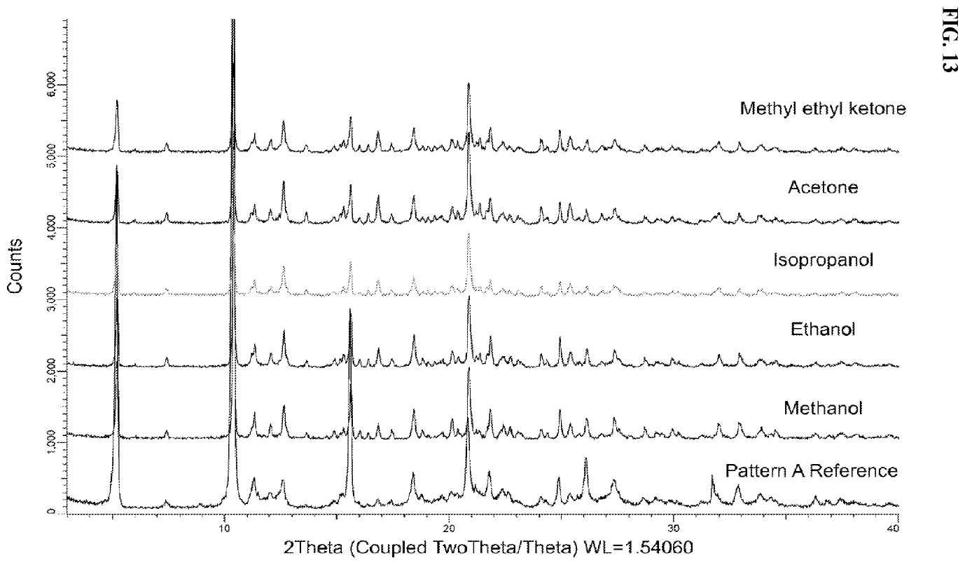

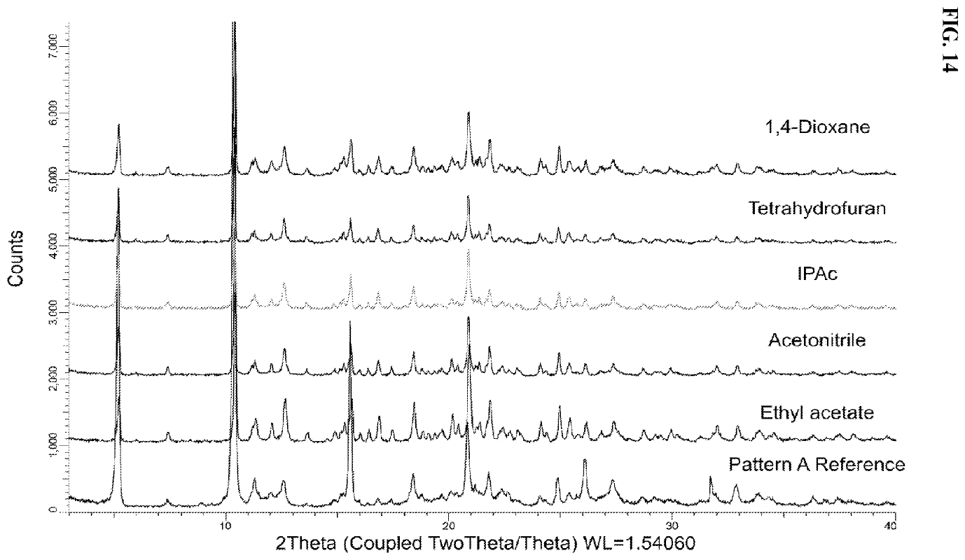

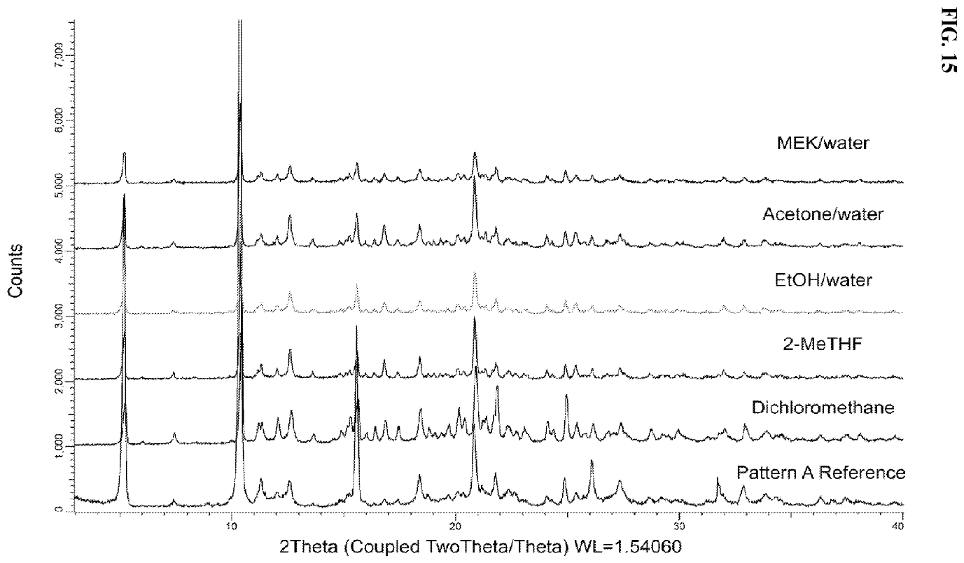

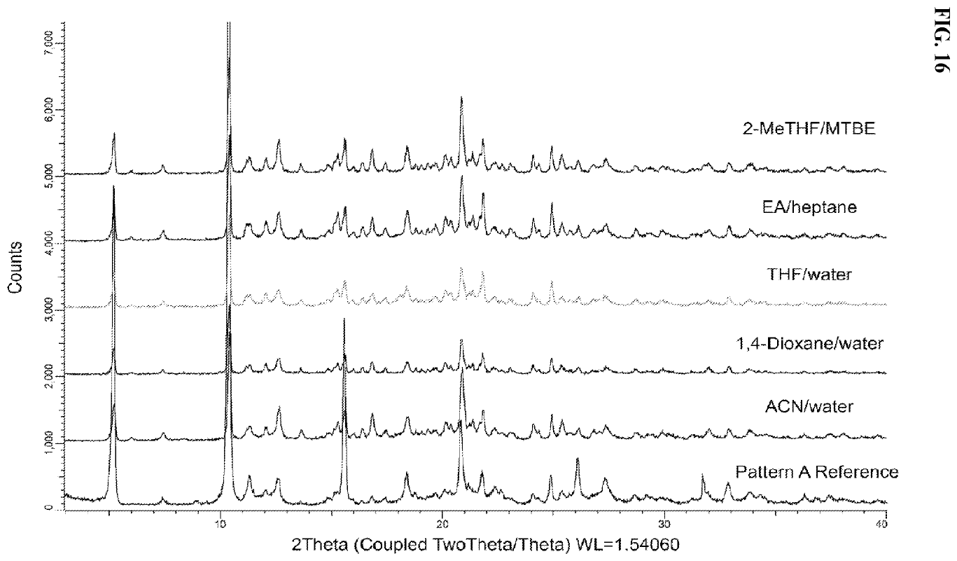

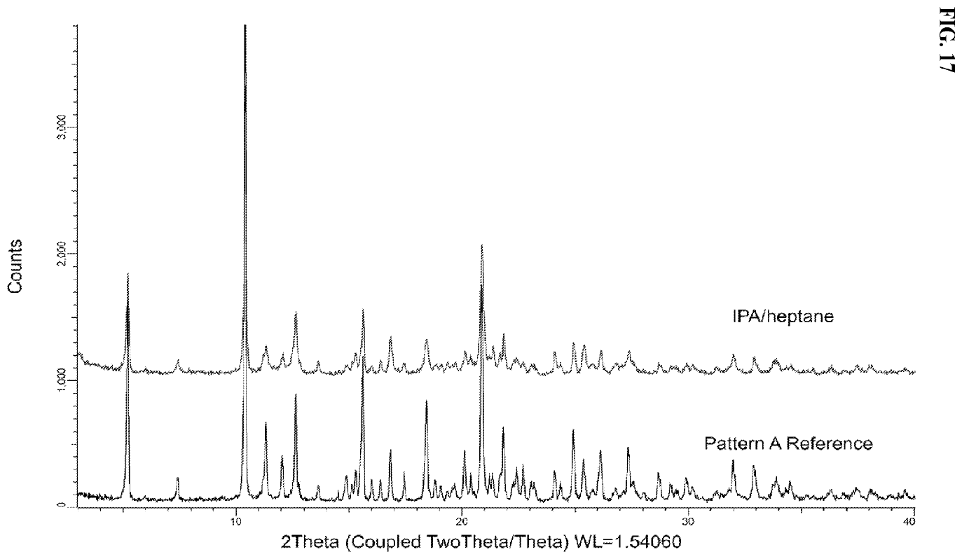

TABLE 2
Summary of equilibration experiments conducted at 25° C. for 2 weeks

| Solvent | XRPD (FIG. 13, FIG. 14, FIG. 15, FIG. 16, FIG. 17) |
|---|---|
| Methanol | Pattern A |
| Ethanol | Pattern A |
| Isopropanol | Pattern A |
| Acetone | Pattern A |
| Methyl ethyl ketone | Pattern A |
| Ethyl acetate | Pattern A |
| Acetonitrile | Pattern A |
| IPAc | Pattern A |
| Tetrahydrofuran | Pattern A |
| 1,4-Dioxane | Pattern A |
| Dichloromethane | Pattern A |
| 2-MeTHF | Pattern A |
| EtOH/water (50:50, v:v) | Pattern A |
| Acetone/water (40:60, v:v) | Pattern A |
| MEK/water (30:70, v:v) | Pattern A |
| ACN/water (30:70, v:v) | Pattern A |
| 1,4-Dioxane/water (20:80, v:v) | Pattern A |
| THF/water (10:90, v:v) | Pattern A |
| EA/heptane (50:50, v:v) | Pattern A |
| 2-MeTHF/MTBE (50:50, v:v) | Pattern A |
| IPA/heptane (25:75, v:v) | Pattern A |

### Equilibration at 50° C. for 1 Week

Approximately 50 mg of trans-4-(3-(2-(difluoromethoxy)-6-methoxypyridin-3-yl)-1-(2-isoproplphenyl)ureido)cyclohexane-1-carboxylic acid was equilibrated in 0.2-1 mL of solvents at 50° C. for 1 week with a stirring bar on a magnetic stirring plate at a rate of 300-400 rpm.

The obtained suspensions were then filtered through a 0.45 μm nylon membrane filter by centrifugation at 14,000 rpm. Solid parts (wet cakes) were investigated by XRPD. The results are summarized in Table 3.

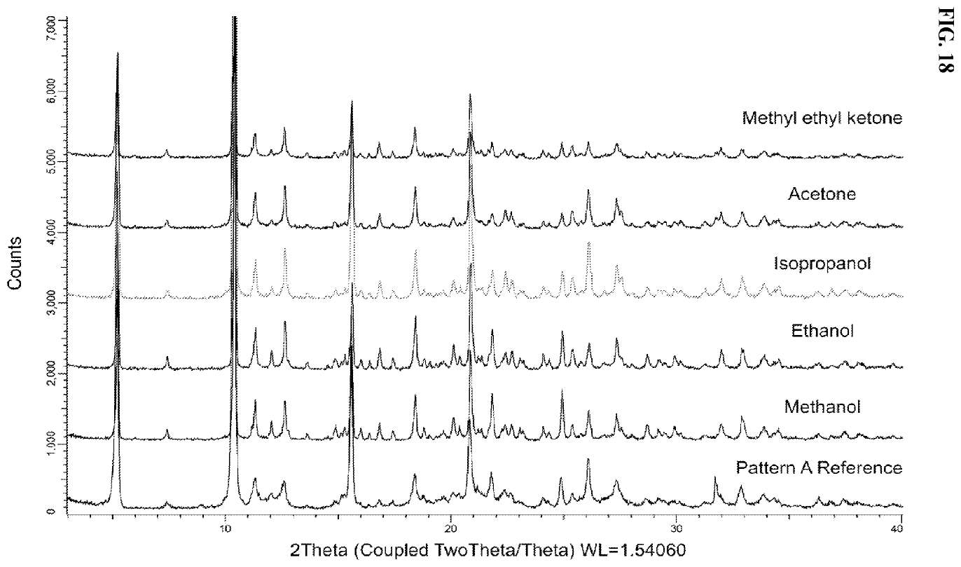

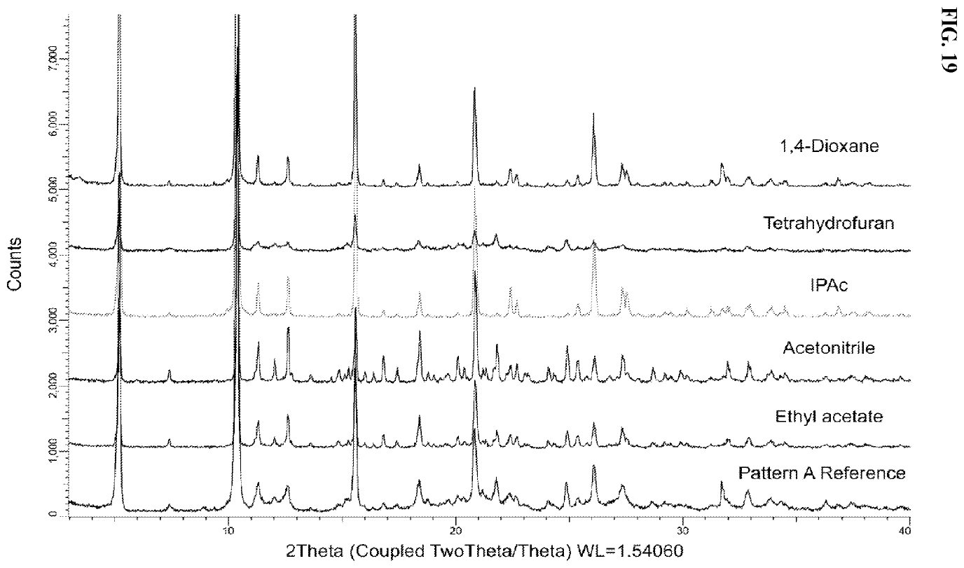

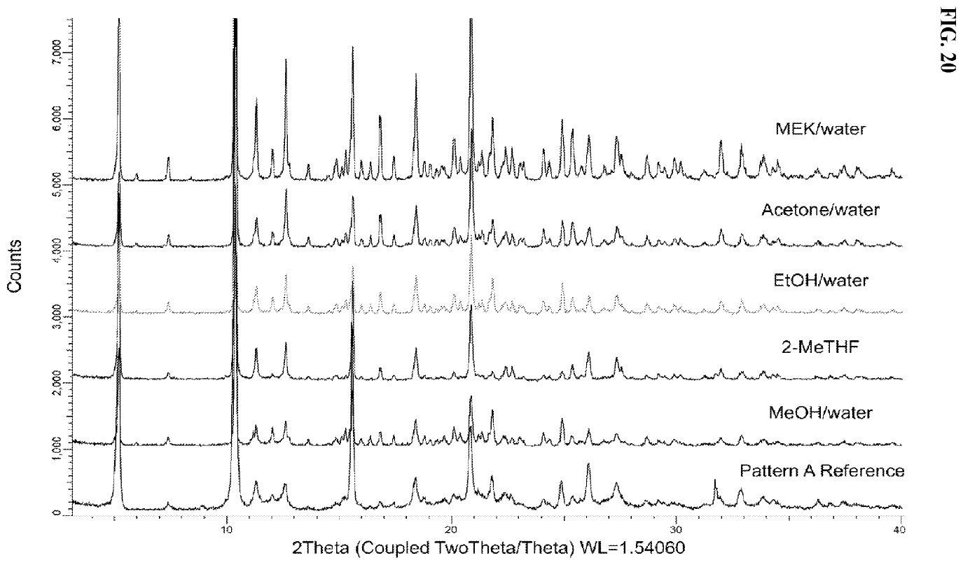

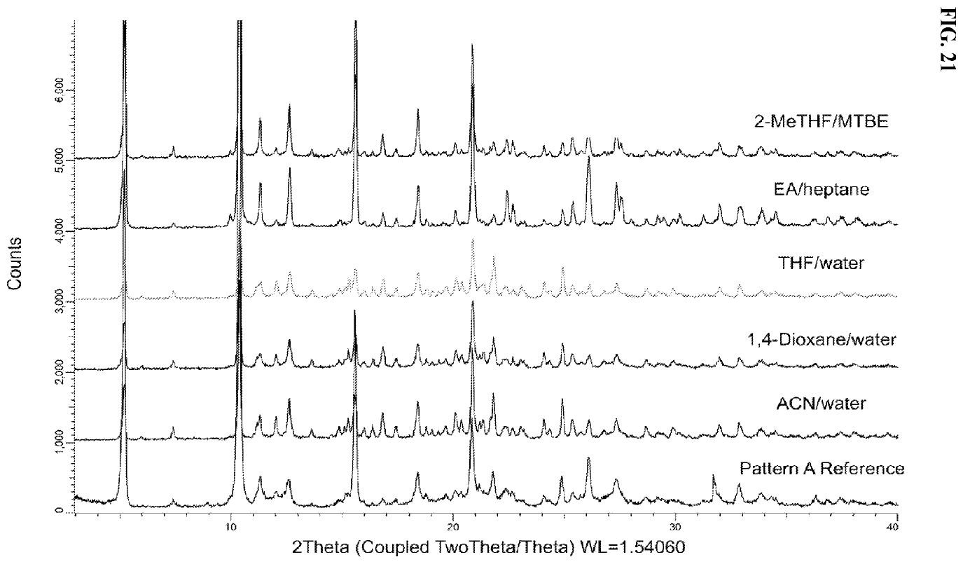

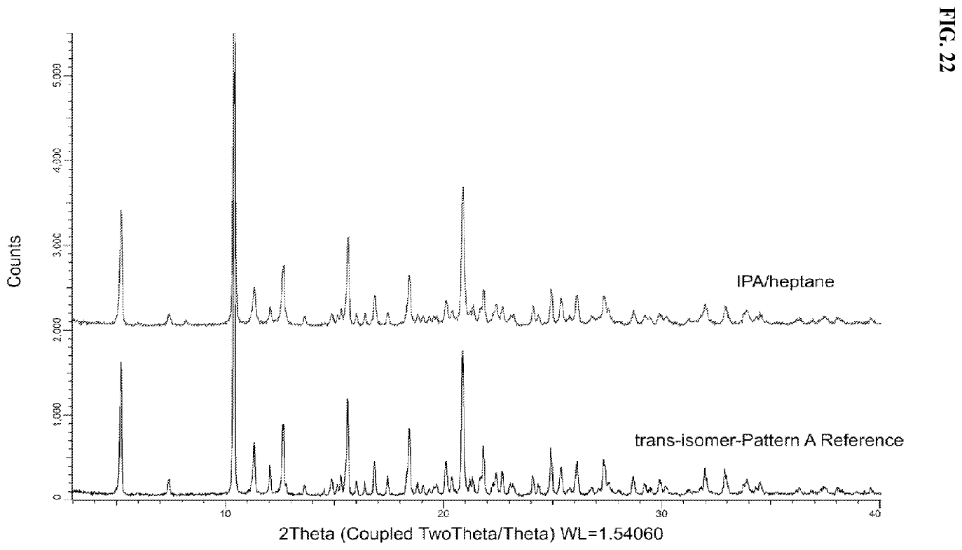

TABLE 3
Summary of equilibration experiments conducted at 50° C. for 1 week

| | XRPD (FIG. 18, FIG. 19, | |
|---|---|---|
| Solvent | FIG. 20, FIG. 21, FIG. 22) | Comments |
| Methanol | Pattern A | n/a |
| Ethanol | Pattern A | n/a |
| Isopropanol | Pattern A | n/a |
| Acetone | Pattern A | n/a |
| Methyl ethyl ketone | Pattern A | n/a |
| Ethyl acetate | Pattern A | n/a |
| Acetonitrile | Pattern A | n/a |
| IPAc | Pattern A | n/a |
| Tetrahydrofuran | Pattern A | n/a |
| 1,4-Dioxane | Pattern A | n/a |
| MeOH/water (20:80, v:v) | Pattern A | n/a |
| 2-MeTHF | Pattern A | n/a |
| EtOH/water (50:50, v:v) | Pattern A | n/a |
| Acetone/water (40:60, v:v) | Pattern A | n/a |
| MEK/water (30:70, v:v) | Pattern A | Agglomeration |
| | | was observed |
| ACN/water (30:70, v:v) | Pattern A | n/a |
| 1,4-Dioxane/water (20:80, v:v) | Pattern A | n/a |
| THF/water (10:90, v:v) | Pattern A | n/a |
| EA/heptane (50:50, v:v) | Pattern A + one small extra | n/a |
| | peak at 10.0°2θ | |
| 2-MeTHF/MTBE (50:50, v:v) | Pattern A + one small extra | n/a |
| | peak at 10.0°2θ | |
| IPA/heptane (25:75, v:v) | Pattern A | n/a |

### Equilibration at 90° C. For 5 Days

Approximately 50 mg of trans-4-(3-(2-(difluoromethoxy)-6-methoxypyridin-3-yl)-1-(2-isopropylphenyl)ureido)cyclohexane-1-carboxylic acid was equilibrated in 0.2-0.6 mL of solvents at 90° C. for 5 days with a stirring bar on a magnetic stirring plate at a rate of 300-400 rpm.

The obtained suspensions were then filtered through a 0.45 μm nylon membrane filter by centrifugation at 14.000 rpm. Solid parts (wet cakes) were investigated by XRPD. The results are summarized in Table 4.

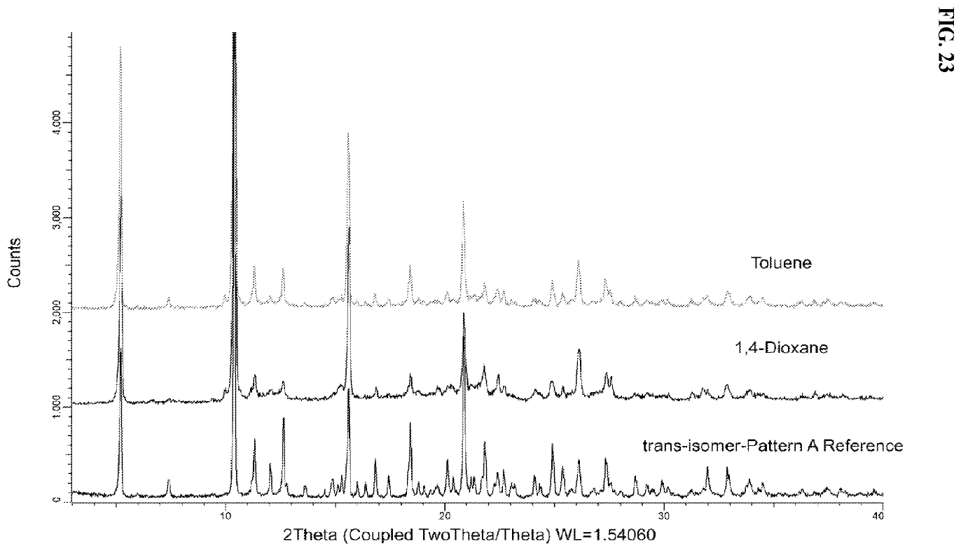

TABLE 4
Summary of equilibration experiments
conducted at 90° C. for 5 days

| Solvent | XRPD (FIG. 23) |
|---|---|
| 1,4-Dioxane | Pattern A + one small extra peak at 10.0°2θ |
| Toluene | Pattern A + one small extra peak at 10.0°2θ |

### Equilibration Under a Temperature Cycle

Approximately 50 mg of trans-4-(3-(2-(difluoromethoxy)-6-methoxypyridin-3-yl)-1-(2-isopropylphenyl)ureido)cyclohexane-1-carboxylic acid was equilibrated in 0.2-1 mL of solvents under a temperature cycle between 5° C. to 50° C. at a heating/cooling rate of 0.1° C./min for 10 cycles. The equilibration was executed with a stirring bar on a magnetic stirring plate at a rate of 400 rpm.

The obtained suspensions were then filtered through a 0.45 μm nylon membrane filter by centrifugation at 14,000 rpm. Solid parts (wet cakes) were investigated by XRPD. The results are summarized in Table 5.

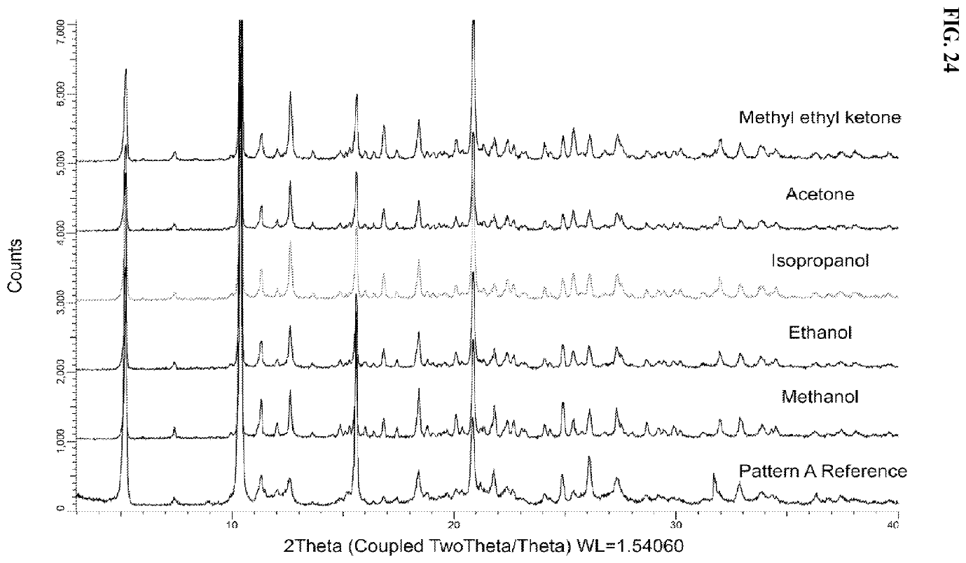

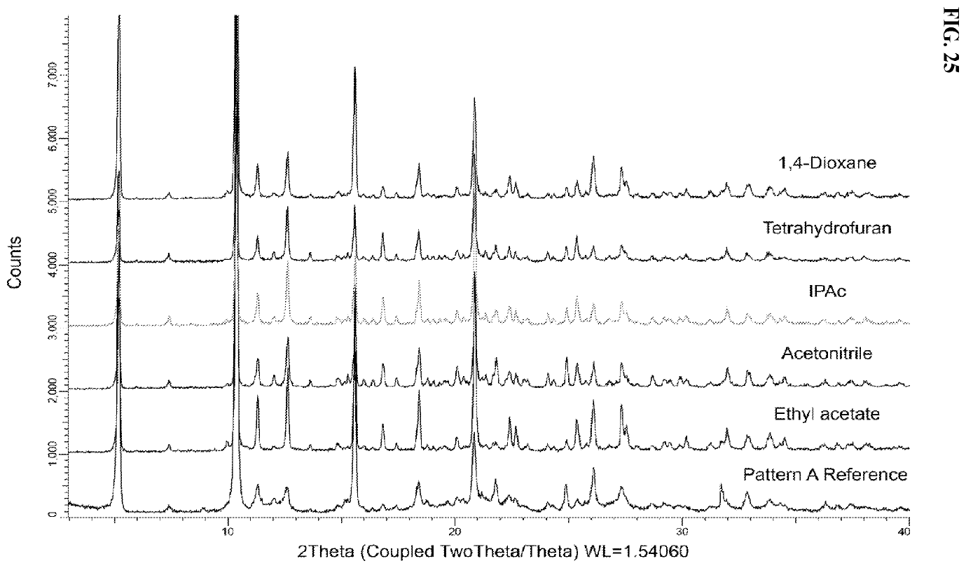

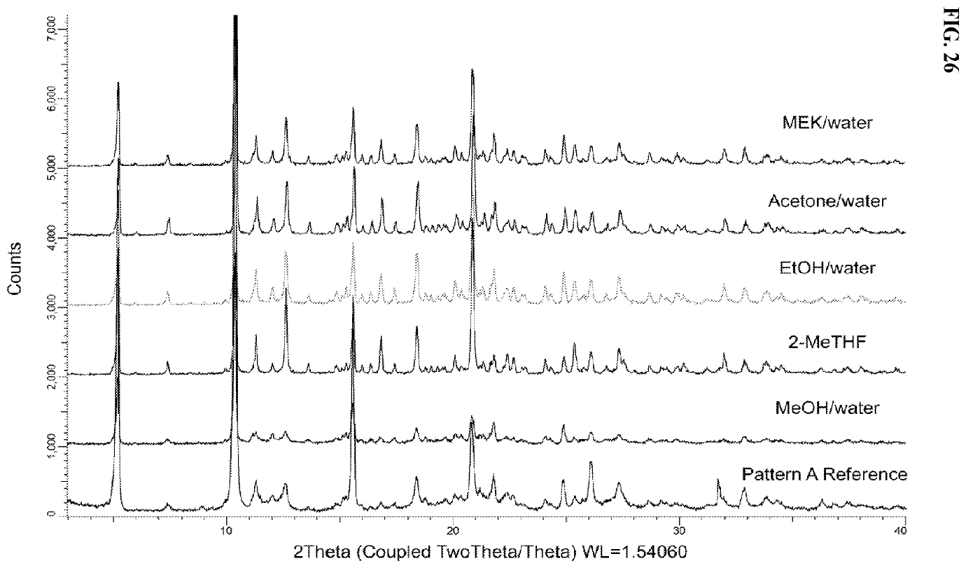

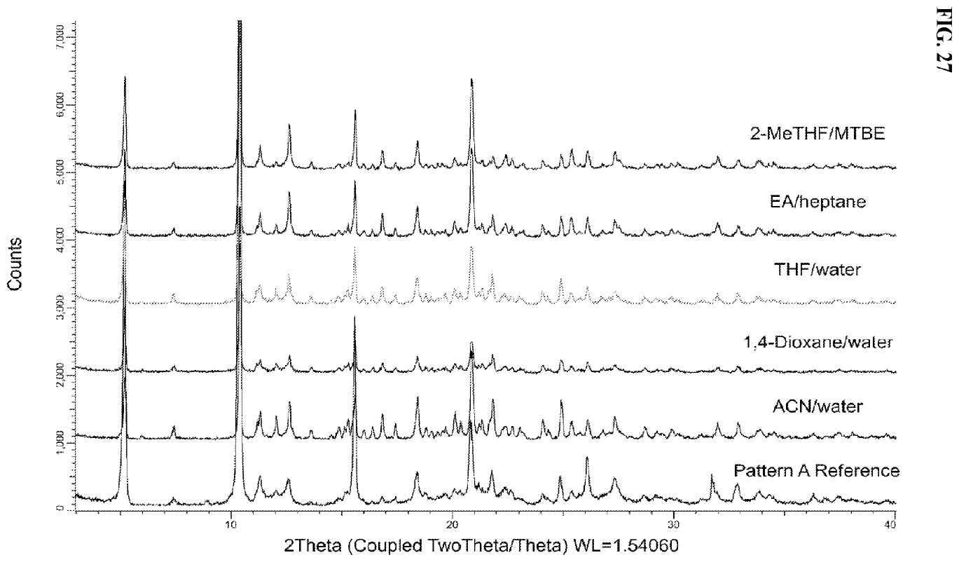

TABLE 5
Summary of equilibration experiments conducted under a temperature cycle

| | XRPD (FIG. 24, FIG. 25, |
|---|---|
| Solvent | FIG. 26, FIG. 27) |
| Methanol | Pattern A + one small extra peak at 10.0°2θ |
| Ethanol | Pattern A + one small extra peak at 10.0°2θ |
| Isopropanol | Pattern A + one small extra peak at 10.0°2θ |
| Acetone | Pattern A |
| Methyl ethyl ketone | Pattern A |
| Ethyl acetate | Pattern A + one small extra peak at 10.0°2θ |
| Acetonitrile | Pattern A + one small extra peak at 10.0°2θ |
| IPAc | Pattern A + one small extra peak at 10.0°2θ |
| Tetrahydrofuran | Pattern A |
| 1,4-Dioxane | Pattern A + one small extra peak at 10.0°2θ |
| MeOH/water (20:80, v:v) | Pattern A |
| 2-MeTHF | Pattern A + one small extra peak at 10.0°2θ |
| EtOH/water (50:50, v:v) | Pattern A + one small extra peak at 10.0°2θ |
| Acetone/water (40:60, v:v) | Pattern A + one small extra peak at 10.0°2θ |
| MEK/water (30:70, v:v) | Pattern A + one small extra peak at 10.0°2θ |
| ACN/water (30:70, v:v) | Pattern A |
| 1,4-Dioxane/water (20:80, v:v) | Pattern A |
| THF/water (10:90, v:v) | Pattern A |
| EA/heptane (50:50, v:v) | Pattern A |
| 2-MeTHF/MTBE (50:50, v:v) | Pattern A |

Crystallization from Hot Saturated Solutions by Slow Cooling

Approximately 30 mg of trans-4-(3-(2-(difluoromethoxy)-6-methoxypyridin-3-yl)-1-(2-isopropylphenyl)ureido)cyclohexane-1-carboxylic acid was dissolved in the minimal amount of selected solvents at 50° C. The obtained solutions were then filtered through a 0.45 μm syringe membrane filter. The clear solutions were cooled to 5° C. at 0.1° C./min. Samples without precipitates at 5° C. were further cooled to −20° C.

Precipitates were collected by centrifugation filtration through a 0.45 μm nylon membrane filter at 14,000 rpm. Solid parts (wet cakes) were investigated by XRPD. The results are summarized in Table 6.

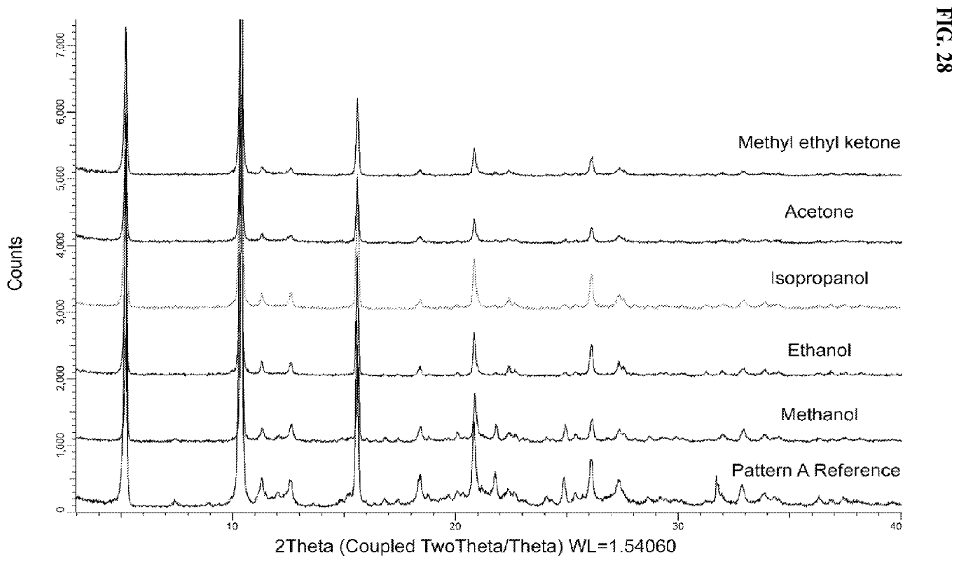

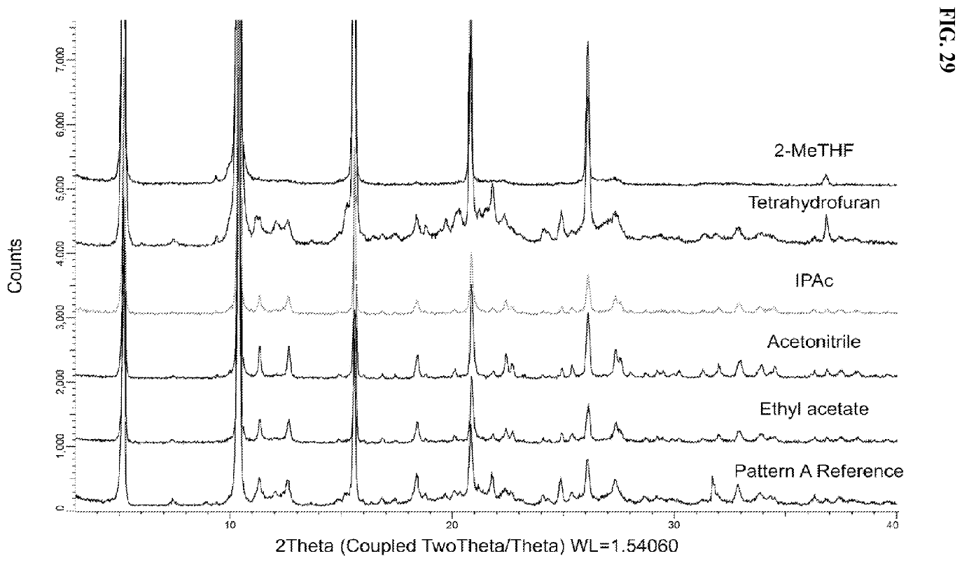

TABLE 6
Summary of crystallization experiments from
hot saturated solutions by slow cooling

| | XRPD (FIG. 28, | |
|---|---|---|
| Solvent | FIG. 29) | Comments |
| Methanol | Pattern A | 5° C.: solids |
| Ethanol | Pattern A | 5° C.: solids |
| Isopropanol | Pattern A | 5° C.: solids |
| Acetone | Pattern A | 5° C.: solids |
| Methyl ethyl ketone | Pattern A | 5° C.: solids |
| Ethyl acetate | Pattern A | 5° C.: solids |
| Acetonitrile | Pattern A | 5° C.: solids |
| IPAc | Pattern A | 5° C.: solids |
| Tetrahydrofuran | Pattern A | 5° C.: clear solution |
| | | Placed at −20° C. for 3 days: solids |
| 2-MeTHF | Pattern A | 5° C.: clear solution |
| | | Placed at −20° C. for 3 days: solids |

Crystallization from Hot Saturated Solutions by Fast Cooling

Approximately 30 mg of trans-4-(3-(2-(difluoromethoxy)-6-methoxypyridin-3-yl)-1-(2-isopropylphenyl)ureido)cyclohexane-1-carboxylic acid was dissolved in the minimal amount of selected solvents at 50° C. The obtained solutions were then filtered through a 0.45 μm syringe membrane filter. The clear solutions were put into a 0° C. ice bath and agitated. Samples without precipitates at 0° C. were further cooled to −20° C.

Precipitates were collected by centrifugation filtration through a 0.45 μm nylon membrane filter at 14,000 rpm. Solid parts (wet cakes) were investigated by XRPD. The results are summarized in Table 7.

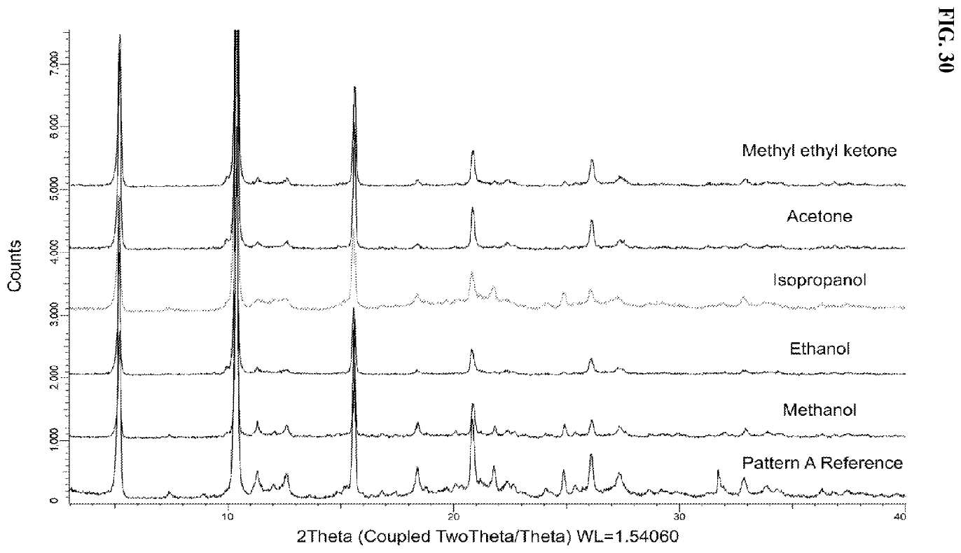

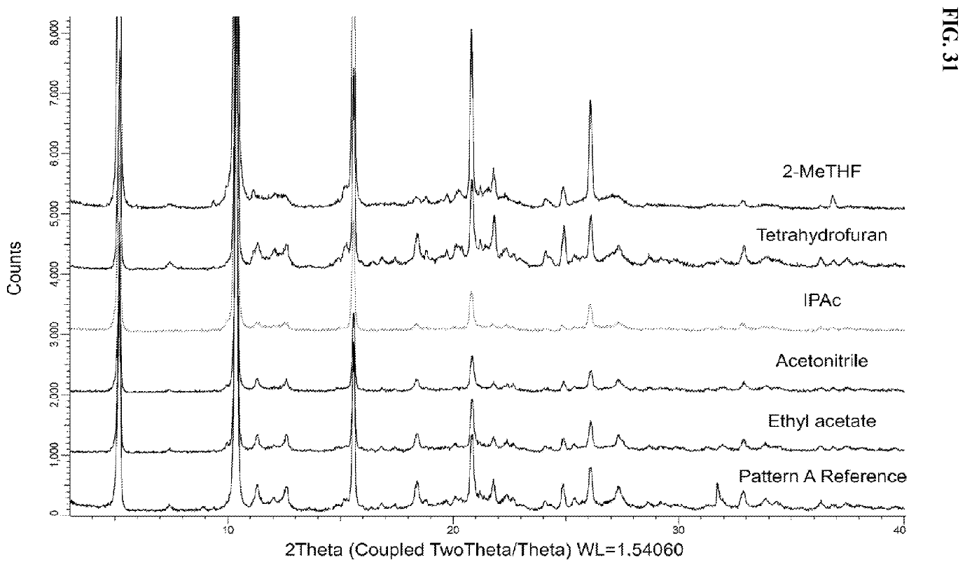

TABLE 7
Summary of crystallization experiments from
hot saturated solutions by fast cooling

| Solvent | XRPD (FIG. 30, FIG. 31) | Comments |
|---|---|---|
| Methanol | Pattern A + one small extra peak | 0° C.: solids |
| | at 10.0°2θ | |
| Ethanol | Pattern A + one small extra peak | 0° C.: solids |
| | at 10.0°2θ | |
| Isopropanol | Pattern A | 0° C.: solids |
| Acetone | Pattern A + one small extra peak | 0° C.: solids |
| | at 10.0°2θ | |
| Methyl ethyl | Pattern A + one small extra peak | 0° C.: solids |
| ketone | at 10.0°2θ | |
| Ethyl acetate | Pattern A + one small extra peak | 0° C.: solids |
| | at 10.0°2θ | |
| Acetonitrile | Pattern A + one small extra peak | 0° C.: solids |
| | at 10.0°2θ | |
| IPAc | Pattern A + one small extra peak | 0° C.: solids |
| | at 10.0°2θ | |
| Tetrahydrofuran | Pattern A | 0° C.: clear solution |
| | | Placed at −20° C. for 4 days: solids |
| 2-MeTHF | Pattern A | 0° C.: clear solution |
| | | Placed at −20° C. for 4 days: solids |

### Crystallization by Slow Addition of an Anti-Solvent to a Homogeneous Solution

Approximately 30 mg of trans-4-(3-(2-(difluoromethoxy)-6-methoxypyridin-3-yl)-1-(2-isopropylphenyl)ureido)cyclohexane-1-carboxylic acid was dissolved in the minimal amount of selected solvents at ambient temperature (about 25° C.). The obtained solutions were then filtered through a 0.45 μm syringe membrane filter. 2-4 Folds of anti-solvent were added into the clear solutions slowly. Samples without precipitates at 25° C. were further cooled to −20° C.

Precipitates were collected by centrifugation filtration through a 0.45 μm nylon membrane filter at 14,000 rpm. Solid parts (wet cakes) were investigated by XRPD. The results are summarized in Table 8.

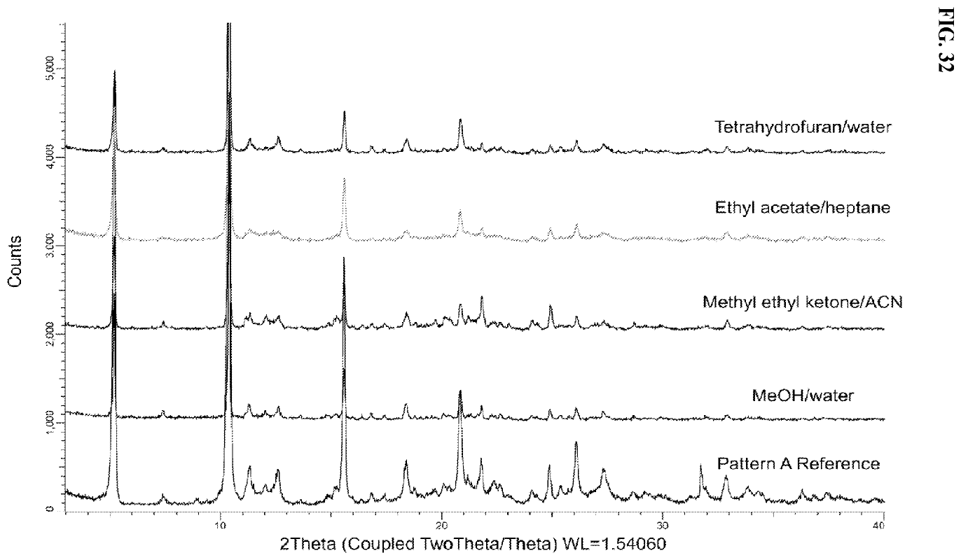

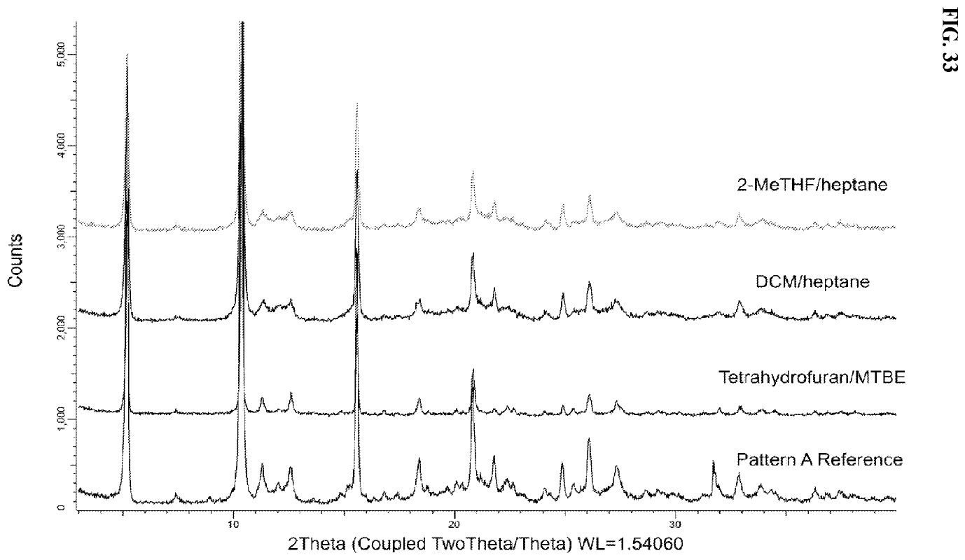

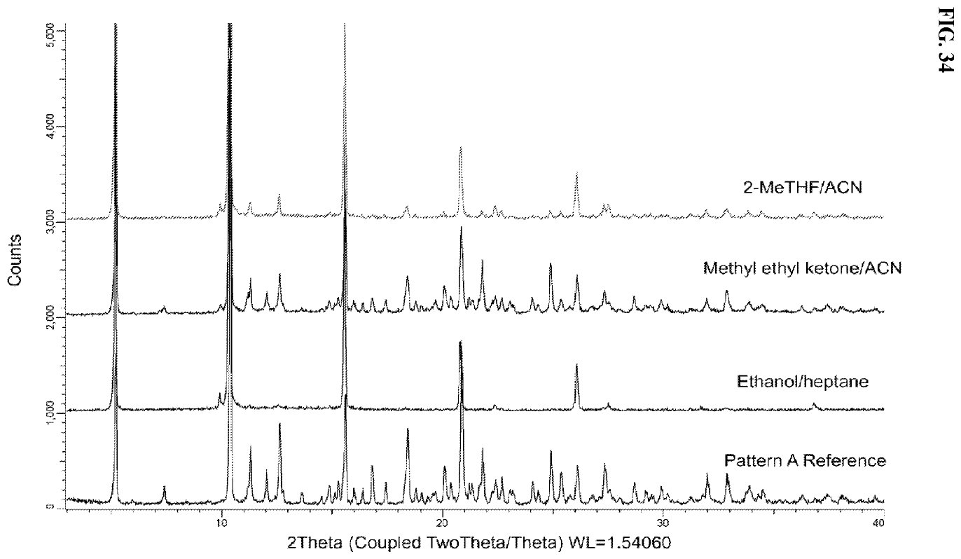

TABLE 8
Summary of crystallization experiments by the slow addition of anti-solvent

| | | XRPD (FIG. 32, | |
|---|---|---|---|
| Solvent (mL) | Anti-solvent (mL) | FIG. 33, FIG. 34) | Comments |
| Methanol (3) | Water (6) | Pattern A | n/a |
| Ethanol (3.4) | Heptane (13.6) | Pattern A + one | 25° C.: clear solution |
| | | small extra peak at | −20° C. for 4 days: solids |
| | | 10.0°2θ | |
| Acetone (1.5) | MTBE (6) | n/a | 25° C.: clear solution |
| | | | −20° C. for 5 days: clear |
| | | | solution |
| Methyl ethyl | ACN (6.6) | Pattern A + one | 25° C.: clear solution |
| ketone (1.65) | | small extra peak at | −20° C. for 5 days: solids |
| | | 10.0°2θ | |
| Methyl ethyl | Water (6.6) | Pattern A | n/a |
| ketone (1.65) | | | |
| Ethyl acetate (3) | Heptane (12) | Pattern A | n/a |
| Tetrahydrofuran | Water (0.4) | Pattern A | n/a |
| (0.2) | | | |
| Tetrahydrofuran | MTBE (0.8) | Pattern A | n/a |
| (0.2) | | | |
| 1,4-Dioxane | MTBE (1.6) | n/a | 25° C.: clear solution |
| (0.4) | | | −20° C. for 5 days: clear |
| | | | solution |
| DCM (1) | Heptane (4) | Pattern A | n/a |
| 2-MeTHF (0.6) | ACN (2.4) | Pattern A + one | 25° C.: clear solution |
| | | small extra peak at | −20° C. for 5 days: solids |
| | | 10.0°2θ | |
| 2-MeTHF (0.6) | Heptane (1.8) | Pattern A | n/a |

### Crystallization by Fast Addition of a Homogeneous Solution to an Anti-Solvent

Approximately 30 mg of trans-4-(3-(2-(difluoromethoxy)-6-methoxypyridin-3-yl)-1-(2-isopropylphenyl)ureido)cyclohexane-1-carboxylic acid was dissolved in the minimal amount of selected solvents at ambient temperature (about 25° C.). The obtained solutions were then filtered through a 0.45 μm syringe membrane filter. The clear solutions were added into 4 folds of anti-solvent quickly. Samples without precipitates at 25° C. were further cooled to −20° C.

Precipitates were collected by centrifugation filtration through a 0.45 μm nylon membrane filter at 14,000 rpm. Solid parts (wet cakes) were investigated by XRPD. The results are summarized in Table 9.

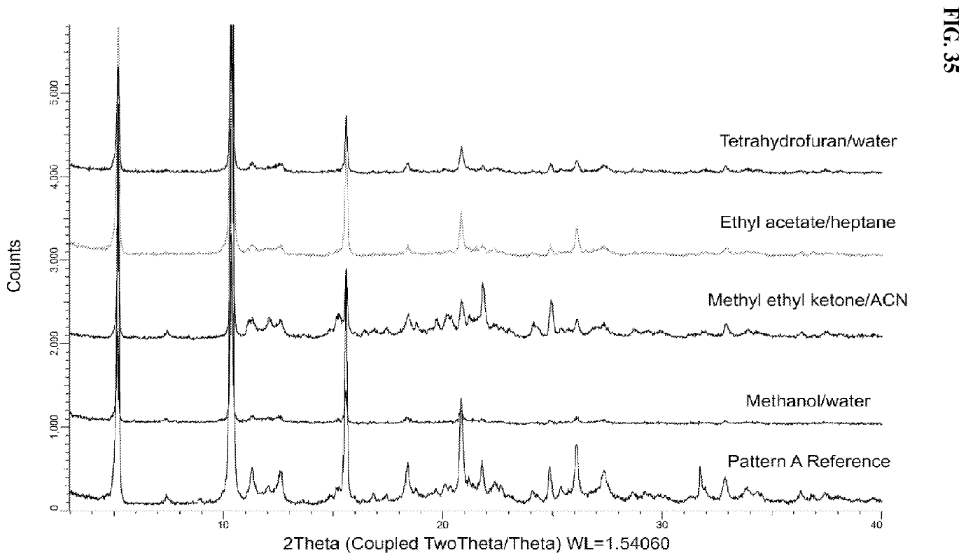

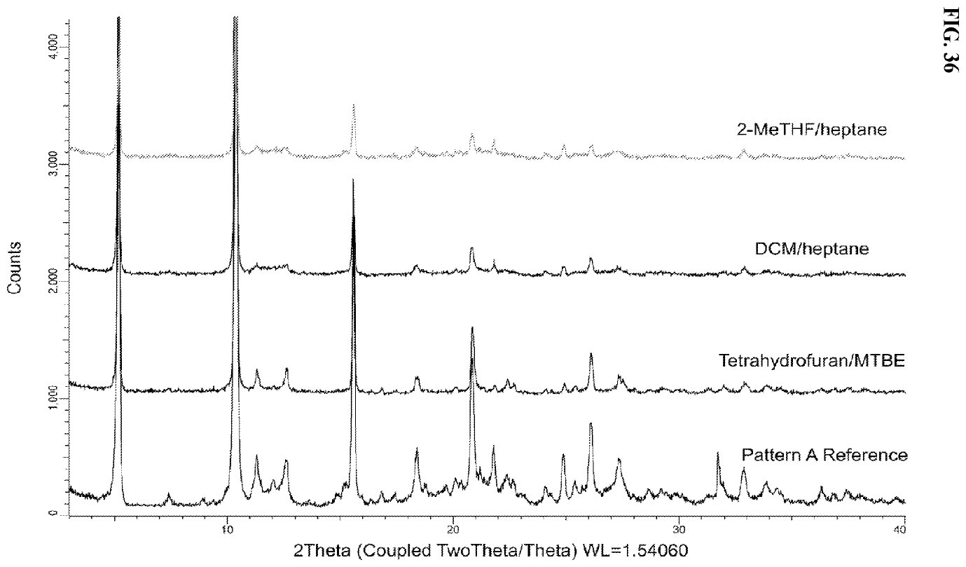

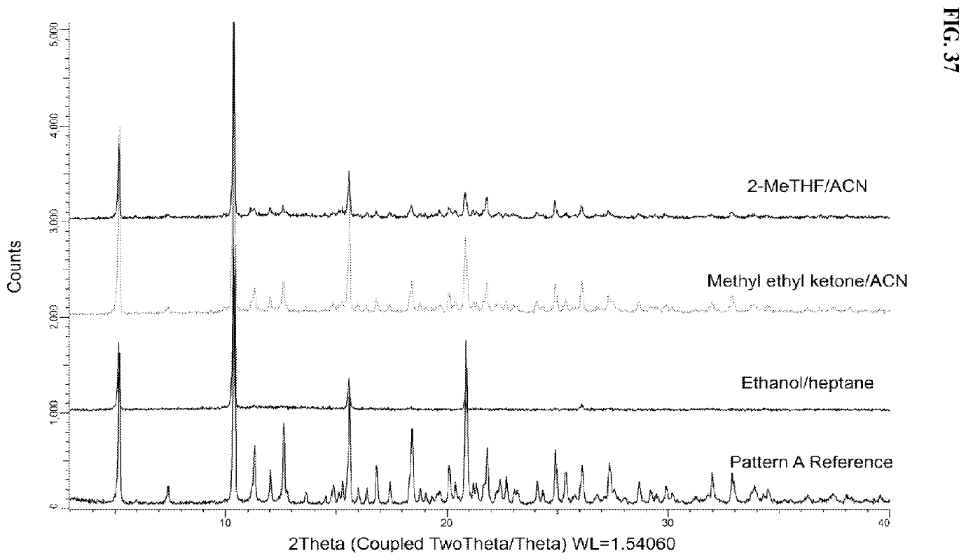

TABLE 9
Summary of crystallization experiments by
the fast reverse addition of anti-solvent

| | | XRPD (FIG. 35, | |
|---|---|---|---|
| Solvent (mL) | Anti-solvent (mL) | FIG. 36, FIG. 37) | Comments |
| Methanol (3) | Water (12) | Pattern A | n/a |
| Ethanol (3.4) | Heptane (13.6) | Pattern A | 25° C.: clear solution |
| | | | −20° C. for 4 days: solids |
| Acetone (1.5) | MTBE (6) | n/a | 25° C.: clear solution |
| | | | −20° C. for 5 days: clear |
| | | | solution |
| Methyl ethyl | ACN (6.6) | Pattern A + one | 25° C.: clear solution |
| ketone (1.65) | | small extra peak at | −20° C. for 4 days: solids |
| | | 10.0°2θ | |
| Methyl ethyl | Water (6.6) | Pattern A | n/a |
| ketone (1.65) | | | |
| Ethyl acetate (3) | Heptane (12) | Pattern A | n/a |
| Tetrahydrofuran | Water (0.8) | Pattern A | n/a |
| (0.2) | | | |
| Tetrahydrofuran | MTBE (0.8) | Pattern A | n/a |
| (0.2) | | | |
| 1,4-Dioxane | MTBE (1.6) | n/a | 25° C.: clear solution |
| (0.4) | | | −20° C. for 5 days: clear |
| | | | solution |
| DCM (1) | Heptane (4) | Pattern A | n/a |
| 2-MeTHF (0.6) | ACN (2.4) | Pattern A | 25° C.: clear solution |
| | | | −20° C. for 4 days: solids |
| 2-MeTHF (0.6) | Heptane (2.4) | Pattern A | n/a |

### Crystallization by Slow Evaporation at Room Temperature

Approximately 30 mg of trans-4-(3-(2-(difluoromethoxy)-6-methoxypyridin-3-yl)-1-(2-isopropylphenyl)ureido)cyclohexane-1-carboxylic acid was dissolved in 0.2-5.0 mL of solvents. The obtained solutions were then filtered through a 0.45 μm syringe membrane filter. The clear solutions were slowly evaporated in ambient condition (about 10-25° C., 20-30% RH).

Solid residues were investigated by XRPD. The results are summarized in Table 10.

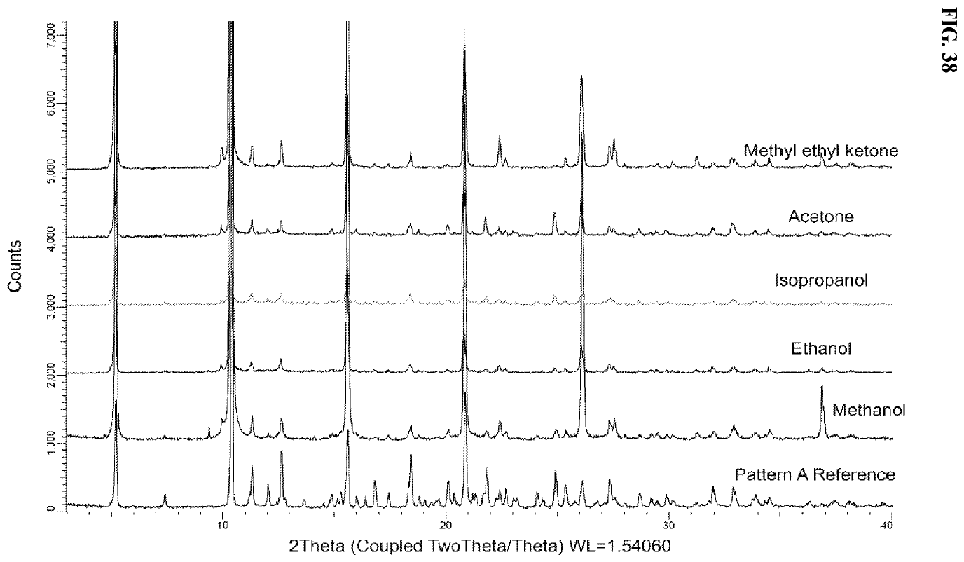

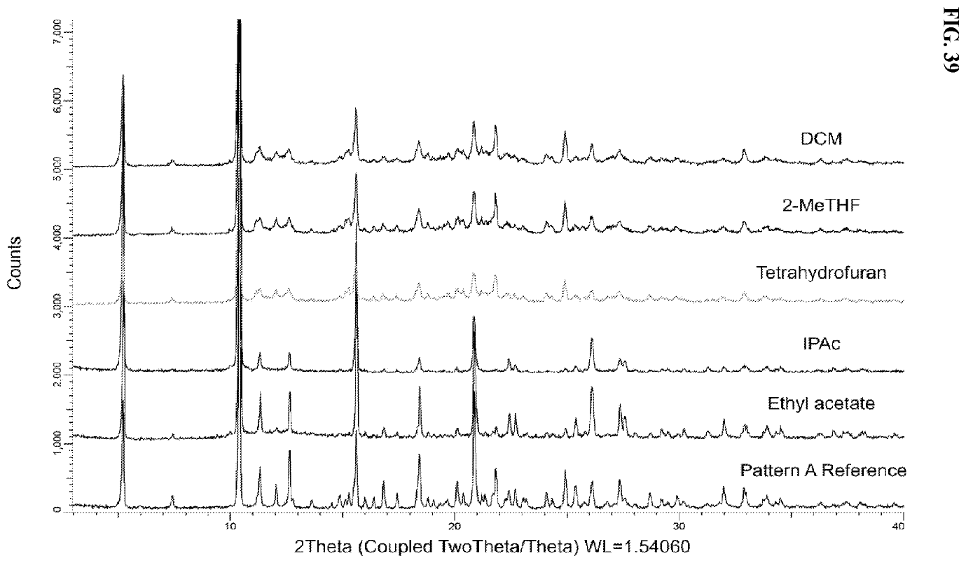

TABLE 10
Summary of crystallization experiments
by slow evaporation at room temperature

| Solvent | XRPD (FIG. 38, FIG. 39) |
|---|---|
| Methanol | Pattern A + one small extra peak at 10.0°2θ |
| Ethanol | Pattern A + one small extra peak at 10.0°2θ |
| Isopropanol | Pattern A + one small extra peak at 10.0°2θ |
| Acetone | Pattern A + one small extra peak at 10.0°2θ |
| Methyl ethyl ketone | Pattern A + one small extra peak at 10.0°2θ |
| Ethyl acetate | Pattern A + one small extra peak at 10.0°2θ |
| IPAc | Pattern A |
| Tetrahydrofuran | Pattern A |
| 2-MeTHF | Pattern A + one small extra peak at 10.0°2θ |
| DCM | Pattern A + one small extra peak at 10.0°2θ |

### Characterization of Crystalline Compound Experiments

### Bulk Stability Determination Experiments

Pattern A crystalline polymorph was placed at 25° C./92% RH in an open container, at 40° C./75% RH in an open container, and at 60° C. in a closed container for 1 week. Samples after the stress were characterized by XRPD and HPLC and inspected for color change. The results are summarized in Table 11.

TABLE 11
Summary of bulk stability experiments under conditions examined

| Initial purity | |
|---|---|
| 99.9% | |
| Purity | Color |

Solid state, 25° C./92% RH, open container, 1 week

| | | |
|---|---|---|
| Bulk (HPLC) | 99.9% | No change |

| | |
|---|---|
| Bulk (XRPD) | Pattern A (FIG. 7) |

Solid state, 40° C./75% RH, open container, 1 week

| | | |
|---|---|---|
| Bulk (HPLC) | 99.9% | No change |

| | |
|---|---|
| Bulk (XRPD) | Pattern A (FIG. 7) |

Solid state, 60° C., tight container, 1 week

| | | |
|---|---|---|
| Bulk (HPLC) | 99.9% | No change |

| | |
|---|---|
| Bulk (XRPD) | Pattern A (FIG. 7) |

### Water Sorption and Desorption Experiments

Water sorption and desorption behavior of Pattern A crystalline polymorph was investigated by DVS at 25° C. with a cycle of 40-0-95-0-40% RH, dm/dt 0.002, minimum equilibration time 60 min, and maximum equilibration time 360 min. XRPD was measured after the DVS test to determine form change. The results are summarized in Table 12.

TABLE 12
Summary of water sorption and desorption experiments

| | Method | | | |
|---|---|---|---|---|
| | 40-0-95-0-40% RH, dm/dt 0.002, minimum equilibration time 60 min, | | | |
| | maximum equilibration time 360 min, 25° C. (FIGS. 8A-8B) | | | |
| Relative | 1^{st} desorp. | 1^{st} sorp. | 2^{nd} desorp. | 2^{nd} sorp. |
| humidity | Weight % | Weight % | Weight % | Weight % |
| at 25° C. | change | change | change | change |
| 0% | 0.03 | 0.03 | 0.00 | 0.00 |
| 10% | 0.05 | 0.05 | 0.02 | 0.02 |
| 20% | 0.05 | 0.05 | 0.03 | 0.02 |
| 30% | 0.06 | 0.05 | 0.03 | 0.02 |
| 40% | 0.06 | 0.05 | 0.04 | 0.03 |
| 50% | n/a | 0.06 | 0.35 | n/a |
| 60% | n/a | 0.06 | 0.64 | n/a |
| 70% | n/a | 0.07 | 0.84 | n/a |
| 80% | n/a | 1.15 | 1.16 | n/a |
| 90% | n/a | 1.93 | 1.93 | n/a |
| 95% | n/a | 3.06 | 3.06 | n/a |

XRPD after DVS test

No form change (FIG. 9)

### Compression Simulation Experiments

About 10 mg of Pattern A crystalline polymorph was compressed for 5 minutes under 5 MPa and 10 MPa with a hydraulic press. Potential form change and degree of crystallinity were evaluated by XRPD. The results are summarized in Table 13.

TABLE 13
Summary of compression simulation experiments

| Pressure | XRPD (FIG. 10) | Comments |
|---|---|---|
| 5 MPa | Pattern A | Slight decrease in crystallinity. |
| | | Peaks becoming broader. |
| 10 MPa | Pattern A | Slight decrease in crystallinity. |
| | | Peaks becoming broader. |

### Dry Grinding Simulation Experiments

About 20 mg of Pattern A crystalline polymorph was ground manually with a mortar and pestle for 1, 3, and 5 min. Potential form change and degree of crystallinity were evaluated by XRPD. The results are summarized in Table 14.

TABLE 14
Summary of dry grinding simulation experiments

| Grinding time | XRPD (FIG. 11) | Comments |
|---|---|---|
| 1 min | Pattern A | Significant decrease in |
| | | crystallinity. |
| | | Peaks becoming broader. |
| | | Partial amorphization. |
| 3 min | Almost amorphous | n/a |
| | form | |
| 5 min | Almost amorphous | n/a |
| | form | |

### Wet Granulation Simulation Experiments

Water or ethanol was added drop wise to about 20 mg of Pattern A crystalline polymorph until the sample was wetted sufficiently. Wet sample was ground gently with a mortar and pestle. Post granulation sample was dried under ambient condition for 3 min. Potential form change and degree of crystallinity were evaluated by XRPD. The results are summarized in Table 15.

TABLE 15
Summary of wet granulation simulation experiments

| Granulation solvent | XRPD (FIG. 12) | Comments |
|---|---|---|
| Water | Pattern A | Significant decrease in |
| | | crystallinity. |
| | | Peaks becoming broader. |
| | | Partial amorphization. |
| Ethanol | Pattern A | Significant decrease in |
| | | crystallinity. |
| | | Peaks becoming broader. |
| | | Partial amorphization. |

### Preparation of Polymorphs

### Preparation of Pattern A

1.0 g of trans-4-(3-(2-(difluoromethoxy)-6-methoxypyridin-3-yl)-1-(2-isopropylphenyl)ureido)cyclohexane-1-carboxylic acid (vide infra) was weighed into a 40 mL glass vial. 10 mL of ACN was added into the vial under stirring at 50° C. for about 1 minute to obtain a suspension.

After stirring at 50° C. for 4 days, solids were collected through a 0.45 μm nylon membrane filter by centrifugation at 4,000 rpm and then dried at 25° C. under vacuum for about 20 hours.

748 mg of Pattern A was obtained as an off-white solid in 76% yield.

### Preparation of trans-4-(3-(2-(difluoromethoxy)-6-methoxypyridin-3-yl)-1-(2-isopropylphenyl)ureido)cyclohexane-1-carboxylic acid

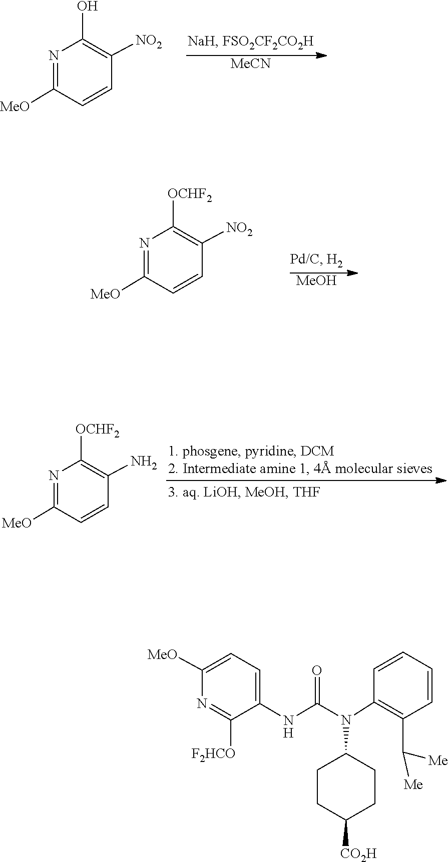 [SMILES: "MeCN" ; "MeOH" ; COc1ccc([N+](=O)[O-])c(O)n1 ; COc1ccc([N+](=O)[O-])c(OC(F)F)n1 ; COc1ccc(N)c(OC(F)F)n1 ; COc1ccc(NC(=O)N(c2ccccc2C(C)C)[C@H]2CC[C@H](C(=O)O)CC2)c(OC(F)F)n1]

Step 1: In a dried, round-bottom flask equipped with a magnetic stirrer was suspended 6-methoxy-3-nitropyridin-2-ol (1 equiv, Combi-Blocks) in acetonitrile (0.10 M). To this was then added sodium hydride (60% w/w dispersion in paraffin oil, 2.8 equiv, Sigma-Aldrich) in one rapid portion and the resulting mixture was stirred at RT for 10 min to afford a brownish, yellow suspension. Then, 2,2-difluoro-2-(fluorosulfonyl)acetic acid (1.8 equiv, Sigma-Aldrich) was added neat and dropwise over a period of 5 min, during which time a mild exotherm was observed. After 16 h of stirring, another aliquot of 2,2-difluoro-2-(fluorosulfonyl)acetic acid (1.8 equiv, Sigma-Aldrich) was added neat and dropwise over a period of 5 min. After another 48 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex:EtOAc) afforded 2-(difluoromethoxy)-6-methoxy-3-nitropyridine as a yellow solid (75% yield).

Step 2: In a dried, round-bottom flask equipped with a magnetic stirrer was dissolved 2-(difluoromethoxy)-6-methoxy-3-nitropyridine (1 equiv) from the previous step in methanol (0.17 M). The resulting yellow solution was then deoxygenated via subsurface purging with nitrogen for 10 min before palladium (10% w/w over activated carbon, dry. 0.08 equiv, Sigma-Aldrich) was added in one rapid portion. The resulting black suspension was then subsurface purged with hydrogen for 10 min before it was stirred under a static hydrogen atmosphere (maintained with a balloon) at RT for 90 min. The reaction was subsequently diluted with EtOAc and filtered through a bed of dichloromethane-wetted celite. The insolubles were washed further with EtOAc. Concentration of the filtrate thus obtained in vacuo afforded 2-(difluoromethoxy)-6-methoxypyridin-3-amine as a reddish, brown solid (>99% yield).

Step 3: In a dried, round-bottom flask equipped with a magnetic stirrer was combined 2-(difluoromethoxy)-6-methoxypyridin-3-amine (1 equiv) from the previous step and pyridine (3 equiv, Sigma-Aldrich) in anhydrous dichloromethane (0.086 M). To this was then added phosgene (15% w/w solution in toluene. 1.5 equiv, Sigma-Aldrich) dropwise at RT and the resulting solution was stirred at RT for 15 min. The volatiles were then removed in vacuo and the crude (2-(difluoromethoxy)-6-methoxypyridin-3-yl)carbamic chloride thus obtained was re-taken up in anhydrous dichloromethane (0.12 M). This solution was then added dropwise at RT to another dichloromethane suspension (0.12 M) of trans-ethyl 4-((2-isopropylphenyl)amino)cyclohexane-1-carboxylate (1 equiv. Intermediate amine 1, vide infra), pyridine (3 equiv, Sigma-Aldrich), and freshly activated 4 Å molecular sieves. The resulting mixture was stirred at RT for 24 h before the reaction was quenched with water. The aqueous layer was separated and back extracted with EtOAc. The combined organic extracts were dried over MgSO₄, filtered, and the filtrate concentrated in vacuo. Purification of the crude product thus obtained by way of column chromatography (SiO₂, gradient elution: Hex→7:3 (v/v) Hex:EtOAc) afforded trans-ethyl 4-(3-(2-(difluoromethoxy)-6-methoxypyridin-3-yl)-1-(2-isopropylphenyl)ureido)cyclohexane-1-carboxylate as a white foam (81% yield).

Step 4: In a round-bottom flask equipped with a magnetic stirrer was dissolved trans-ethyl 4-(3-(2-(difluoromethoxy)-6-methoxypyridin-3-yl)-1-(2-isopropylphenyl)ureido)cyclohexane-1-carboxylate (1 equiv) from the previous step in a 1:1 (v/v) solution of THF and methanol (0.11 M). To this colorless solution was then added lithium hydroxide (1 M solution in water, 5 equiv) and the resulting mixture was heated at 45° C. for 3 h. Upon cooling to RT, the reaction mixture was carefully quenched with HCl (1 M solution in water, 5 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded a white solid that was then vigorously stirred in hot isopropanol until homogeneous. The solution thus obtained was then allowed to cool until cloudy, at which time an equal volume of heptane was added with vigorous stirring. After 16 h of stirring at RT, the suspension was filtered to afford the title compound as a white solid.

LCMS: m/z=478.1 [M+H]⁺; ¹H NMR (DMSO-d₆): δ=8.08 (d, J=8.7 Hz, 1H), 7.56~7.49 (m, 2H), 7.36 (t, J=72.9 Hz, 1H), 7.35 (td, J=7.2, 1.8 Hz, 1H), 7.25 (dd, J=8.1, 1.2 Hz, 1H), 6.54 (d, J=8.7 Hz, 1H), 6.29 (s, 1H), 4.30~4.21 (m, 1H), 3.82 (s, 3H), 3.22 (sept, J=6.9 Hz, 1H), 2.24~1.85 (m, 5H), 1.62~1.46 (m, 3H), 1.29 (d, J=6.9 Hz, 3H), 1.22~1.05 (m. 4H).

### Intermediate amine 1: Preparation of trans-ethyl 4-((2-isopropylphenyl)amino)cyclohexane-1-carboxylate

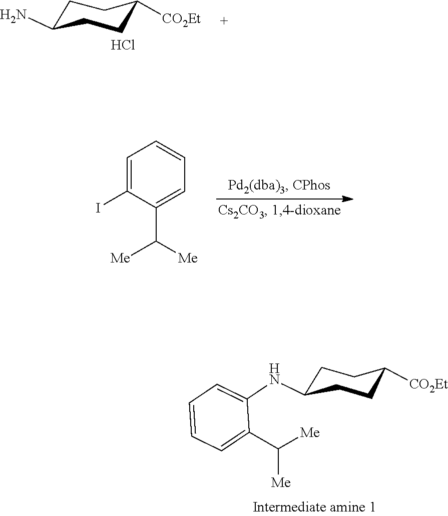

In a thick-walled glass reaction vessel equipped with a magnetic stirrer and a Teflon screwcap was combined trans-ethyl 4-aminocyclohexanecarboxylate hydrochloride (1 equiv, ChemScene), 1-iodo-2-isopropylbenzene (1.4 equiv, Combi-Blocks), tris(dibenzylidineacetone)dipalladium(0) (0.1 equiv, Sigma-Aldrich), 2-cyclohexylphosphino-2′,6′-bis(N,N-dimethylamino)biphenyl (0.2 equiv, Combi-Blocks), and cesium carbonate (4 equiv, Sigma-Aldrich) in 1,4-dioxane (0.12 M). The resulting purple suspension was then deoxygenated via subsurface purging with nitrogen for 10 min before the reaction vessel was tightly sealed and heated at 90° C. for 48 h. The resulting orange, brown suspension was cooled to RT, diluted with tert-butyl methyl ether, and washed sequentially with water and brine. The organic extract thus obtained was then dried over MgSO₄, treated with charcoal, filtered through a bed of celite, and the filtrate concentrated in vacuo. Purification of the crude product thus obtained by way of column chromatography (SiO₂, gradient elution: Hex→4:1 (v/v) Hex:EtOAc) afforded the title compound as a golden yellow oil (67% yield).

### Biological Evaluations: In Vitro Functional Assay of Lysophosphatidic Acid Receptor 1 Activity

Primary compound plates were prepared in 100% DMSO (Sigma-Aldrich), secondary compound plates were prepared at 10× concentration in DMEM (Invitrogen), and tertiary compound plates were prepared at 3× concentration in assay buffer containing HBSS (no Ca⁺²/Mg⁻². Invitrogen) and 0.1% BSA (Sigma-Aldrich). Fluo-4 NW calcium assay dye (Invitrogen) was prepared as per manufacturer's recommendations in assay buffer. B103 cells stably expressing human LPAR1 (J. Chun lab, UCSD) were grown to confluency in DMEM media (Invitrogen) containing 10% FBS (ATCC), 10% Penicillin-Streptomycin (Sigma-Aldrich) and 50 μg Geneticin (Sigma-Aldrich), and detached with Accutase (Sigma-Aldrich) prior to assay. The freshly detached cells were resuspended in growth media and plated in black, clear-bottom 96-well plates (Costar) containing compound (i.e., secondary compound plate) at a density of 5×10⁴ cells/well. Once plated, cells were left at room temperature for 30 minutes and then transferred to a 37° C. 5% CO₂ incubator for 24 hours. After 24 hours, the growth media was then removed and freshly prepared Fluo-4 NW calcium assay dye was added to cells. Compounds (i.e., tertiary compound plates) were then added back to the dye/cells, returned to the incubator for 30 minutes, and then kept at room temperature for an additional 30 minutes. Finally, lysophosphatidic acid (18:1) at the EC₈₀ concentration was added and calcium flux measured using FlexStation 3 (Molecular Devices). Sigmoidal dose-response curves were generated by measuring luminescence over 45 sec and calculating the area under the curve. Dose response curves and IC₅₀ values were generated using Prism (GraphPad). Compounds were tested at a final concentration range of 100 pM to 10 μM in 0.10% DMSO.

The lysophosphatidic acid receptor 1 IC₅₀ for trans-4-(3-(2-(difluoromethoxy)-6-methoxypyridin-3-yl)-1-(2-isopropylphenyl)ureido)cyclohexane-1-carboxylic acid was found to be 10 nM.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A crystalline compound having the formula: 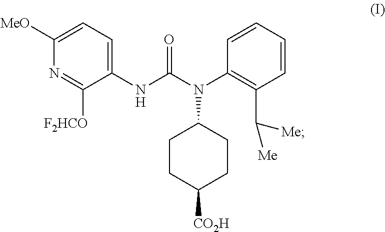 [SMILES: COc1ccc(NC(=O)N(c2ccccc2C(C)C)[C@H]2CC[C@H](C(=O)O)CC2)c(OC(F)F)n1]
wherein the crystalline compound has characteristic X-ray powder diffraction peaks at about 5.2° 2θ, about 10.4° 2θ, and about 15.6° 2θ.

2. The crystalline compound of claim 1, wherein the crystalline compound has further characteristic X-ray powder diffraction peaks at about 11.3° 2θ, about 12.6° 2θ, about 18.4° 2θ, about 20.9° 2θ, about 22.4° 2θ, about 26.1° 2θ, about 27.4° 2θ, and about 27.6° 2θ.

3. The crystalline compound of claim 1, wherein the crystalline compound has further characteristic X-ray powder diffraction peaks at about 6.7° 2θ, about 7.3° 2θ, about 8.6° 2θ, about 9.4° 2θ, about 11.3° 2θ, about 12.0° 2θ, about 12.6° 2θ, about 13.6° 2θ, about 14.9° 2θ, about 16.8° 2θ, about 17.4° 2θ, about 18.4° 2θ, about 18.8° 2θ, about 19.5° 2θ, about 20.1° 2θ, about 20.9° 2θ, about 21.8° 2θ, about 22.4° 2θ, about 22.7° 6 2θ, about 23.1° 2θ, about 24.1° 2θ, about 24.4° 2θ, about 24.9° 2θ, about 25.4° 2θ, about 26.1° 2θ, about 27.4° 2θ, about 27.6° 2θ, about 28.0° 2θ, about 28.4° 2θ, about 28.7° 2θ, about 29.2° 2θ, about 29.5° 2θ, about 30.0° 2θ, about 30.2° 2θ, about 31.3° 2θ, about 31.8° 2θ, about 32.0° 2θ, about 32.9° 2θ, about 33.9° 2θ, about 34.5° 2θ, about 36.3° 2θ, about 36.9° 2θ, about 37.5° 2θ, about 38.0° 2θ, and about 39.6° 2θ.

4. The crystalline compound of claim 1, wherein the crystalline compound is further characterized as having a differential scanning calorimetry endotherm onset at about 188.6° C.

5. A pharmaceutical composition comprising the crystalline compound of one of claims 1 to 4 and a pharmaceutically acceptable excipient.

6. The pharmaceutical composition of claim 5, wherein the pharmaceutical composition does not comprise the compound of formula I in a crystalline form other than said crystalline compound.

7. A method of treating a neurodegenerative disorder in a subject in need thereof, said method comprising administering to the subject in need thereof a therapeutically effective amount of the crystalline compound of one of claims 1 to 4.

8. A method of treating an inflammatory disease in a subject in need thereof, said method comprising administering to the subject in need thereof a therapeutically effective amount of the crystalline compound of one of claims 1 to 4.

9. The method of claim 8, wherein the inflammatory disease is encephalitis.

10. The method of claim 9, wherein the encephalitis is post-hemorrhagic encephalitis.

11. A method of treating a demyelinating disease in a subject in need thereof, said method comprising administering to the subject in need thereof a therapeutically effective amount of the crystalline compound of one of claims 1 to 4.

12. The method of claim 11, wherein the demyelinating disease is a demyelinating disease of the central nervous system.

13. The method of claim 12, wherein the demyelinating disease is multiple sclerosis.

14. The method of claim 11, wherein the demyelinating disease is a demyelinating disease of the peripheral nervous system.

15. A method of treating a fibrotic disease in a subject in need thereof, said method comprising administering to the subject in need thereof a therapeutically effective amount of the crystalline compound of one of claims 1 to 4.

16. The method of claim 15, wherein the fibrotic disease is pulmonary fibrosis, skin fibrosis, liver fibrosis, or ocular fibrosis.

17. The method of claim 15, wherein the fibrotic disease is idiopathic pulmonary fibrosis, scleroderma, nonalcoholic steatohepatitis, or ocular fibrosis.

18. A method of treating cancer in a subject in need thereof, said method comprising administering to the subject in need thereof a therapeutically effective amount of the crystalline compound of one of claims 1 to 4.

19. The method of claim 18, wherein the cancer is brain cancer.

20. The method of claim 19, wherein the cancer is glioblastoma.

21. A method of treating an LPAR1-associated disease in a subject in need thereof, said method comprising administering to the subject in need thereof a therapeutically effective amount of the crystalline compound of one of claims 1 to 4, wherein the LPAR1-associated disease is neuropathic pain.

22. A method of modulating LPAR1 activity in a subject, said method comprising administering to the subject the crystalline compound of one of claims 1 to 4.
